(19) United States
(12) Patent Application Publication
(10) Pub. No.: US 2023/0079226 A1
(43) Pub. Date: Mar. 16, 2023

(54) A METHOD OF DETERMINING A STRUCTURE OF RIBONUCLEIC ACID MOLECULES AND RELATED KITS

(71) Applicant: AGENCY FOR SCIENCE, TECHNOLOGY AND RESEARCH, Singapore (SG)

(72) Inventors: Jiaxu WANG, Singapore (SG); Yue WAN, Singapore (SG)

(21) Appl. No.: 17/797,950

(22) PCT Filed: Feb. 10, 2021
(86) PCT No.: PCT/SG2021/050070

§ 371 Date: Aug. 5, 2022

(30) Foreign Application Priority Data
Feb. 10, 2020 (SG) 10202001192W

(51) Int. Cl. C12Q 1/6806; C12Q 1/6869

(52) U.S. Cl. CPC C12Q 1/6806; C12Q 1/6869; C12Q 2521/107; C12Q 2527/101; C12Q 2527/125

(57) ABSTRACT

The invention relates to a method of determining a structure of ribonucleic acid (RNA) molecules, the method comprising: contacting the RNA molecules with a modifying agent to obtain modified RNA molecules; reverse transcribing the modified RNA molecules; sequencing the product obtained from the preceding step to generate sequencing reads; and analysing the sequencing reads to determine the structure of the RNA molecules. In a preferred embodiment, the RNA molecules consist of RNA molecules of a single cell, and the modifying agent is 2-methylnicotinic acid imidazolide-azide (NAI-N3). There is also provided a method of classifying cells into one or more cell populations, the method comprising: determining the structure of RNA molecules in each cell; and classifying the cells into one or more cell populations based on similarity in the structure of their RNA molecules.

### TECHNICAL FIELD

The present disclosure relates broadly to a method of determining a structure of ribonucleic acid (RNA) molecules and related kits and methods thereof.

### BACKGROUND

Beyond its sequence, RNAs contain a secondary level of information at the level of structure. RNA structures can provide important gene regulatory information across different cell types. Knowledge of the structural characteristics of RNAs therefore allows for a better understanding of RNA functions and mechanisms of action.

The structural characteristics of RNAs can be determined by RNA structure probing coupled to high throughput sequencing. However, as structural information is aggregated across millions of cells being used as the starting material in this approach, structural heterogeneity that is present in individual cells is lost.

The typical RNA structure probing procedure is unable to provide RNA structural information at a single-cell resolution. In a typical RNA structure probing procedure, chemical probes are used to detect and modify single-stranded (i.e., unpaired) bases along an RNA. These chemical probes preferentially react with single-stranded bases to form modified bases. The modified RNA is then reverse transcribed into cDNA. During reverse transcription, reverse transcriptase (RT) enzymes are sometimes blocked by the modifications, while at other times, under certain chemical conditions, the RT enzymes “jump” through the modifications and incorporate an erroneous base or a mutation. The fraction of mutations at a particular base can be calculated as an approximate for the likelihood of single-strandedness at that base, providing structural information along the RNA. However, the low “jump” through rates in a typical reverse transcription procedure require that at least 500 reads per base be obtained for accurate structure determination, thus limiting the utility of the procedure for single cell RNA structure determination.

Thus, there is a need to provide an alternative method of determining a structure of RNA and related kits and methods thereof.

### SUMMARY

In one aspect, there is provided a method of determining a structure of ribonucleic acid (RNA) molecules, the method comprising: contacting the RNA molecules with a modifying agent to obtain modified RNA molecules; reverse transcribing the modified RNA molecules; sequencing the product obtained from the preceding step to generate sequencing reads; and analysing the sequencing reads to determine the structure of the RNA molecules.

In one embodiment, the modifying agent comprises 2-methylnicotinic acid imidazolide (NAI) or derivatives thereof.

In one embodiment, the reverse transcribing step is carried out in a manganese-containing medium, optionally wherein the reverse transcribing step is carried out at a temperature of less than 50° C.

In one embodiment, the modifying agent comprises 2-methylnicotinic acid imidazolide (NAI) or derivatives thereof and wherein the reverse transcription step is carried out in a manganese-containing medium.

In one embodiment, the reverse transcribing step is carried out using Moloney murine leukemia virus (MMLV) reverse transcriptase, optionally a genetically modified MMLV reverse transcriptase.

In one embodiment, the reverse transcribing step is carried out for least about 2 hours, optionally at least about 4 hours, further optionally at least about 6 hours, further optionally at least about 8 hours.

In one embodiment, the method further comprises fragmenting the modified RNA molecules prior to the reverse-transcribing step.

In one embodiment, fragmenting the modified RNA molecules comprises heating the modified RNA molecules.

In one embodiment, the modified RNA molecules are heated in the presence of deoxynucleoside triphosphate (dNTP).

In one embodiment, the fragmenting step is carried out in a medium that is substantially free of magnesium.

In one embodiment, the fragmenting step is carried out in the same vessel as the reverse transcribing step.

In one embodiment, the method further comprises amplifying the reverse transcribed product prior to the sequencing step.

In one embodiment, the method further comprises purifying the amplicons, further optionally purifying the amplicons for at least two times.

In one embodiment, the RNA molecules consist of RNA molecules of a single cell.

In one aspect, there is provided a method of simultaneously determining a structure of an RNA molecule of a gene and an expression of the gene, the method comprising: contacting the RNA molecule with a modifying agent to obtain a modified RNA molecule; reverse transcribing the modified RNA molecule; sequencing the product obtained from the preceding step to generate sequencing reads; analysing the sequencing reads to determine the structure of the RNA molecule of the gene; and evaluating the amount of sequencing reads to determine the expression of the gene.

In one aspect, there is provided a method of characterising a cell, the method comprising: determining the structure of RNA molecules in the cell according to embodiments of the method as described herein.

In one aspect, there is provided a method of classifying cells into one or more cell populations, the method comprising: determining the structure of RNA molecules in each cell according to embodiments of the method as described herein; and classifying the cells into one or more cell populations based on similarity in the structure of their RNA molecules.

In one embodiment, the method is a method of classifying cells into different cell types and/or different stages of development.

In one aspect, there is provided a kit for determining a structure of RNA molecules according to embodiments of the method as described herein, the kit comprising: a modifying agent comprising 2-methylnicotinic acid imidazolide (NAI) or derivatives thereof for modifying the RNA molecules; a reverse transcription medium comprising manganese; and optionally, a Moloney murine leukemia virus (MMLV) reverse transcriptase, further optionally a genetically modified MMLV reverse transcriptase.

In one aspect, there is provided a kit for determining a structure of RNA molecules according to embodiments of the method as described herein, the kit comprising: a single medium for fragmentation of the RNA molecules and annealing of a primer to the RNA molecules for reverse transcription, wherein the single medium is substantially free of magnesium, optionally wherein the single medium comprises deoxyribonucleotide triphosphates (dNTPs).

### DEFINITIONS

The term “medium” as used herein broadly refers to a liquid in which one or more materials (e.g., Mn²⁺ ions) may be dispersed or dissolved in.

The term “micro” as used herein is to be interpreted broadly to include dimensions from about 1 micron to about 1000 microns.

The term “nano” as used herein is to be interpreted broadly to include dimensions less than about 1000 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a method of determining/obtaining/predicting/probing a structure or a structural information/data of one or more ribonucleic acid (RNA) or RNA molecules are disclosed hereinafter. Advantageously, embodiments of the method show improved scale and sensitivity and are capable of determining a structure or a structural information of RNA at a single-cell resolution. Embodiments of the method thus allow for the identification of structural heterogeneity, on top of expression heterogeneity. In various examples, an average structure or structural information/data of RNA obtained from single cells by embodiments of the method shows high correlation with the structural information/data of RNA obtained from 10 cells, 100 cells and millions of cells, attesting to the reliability and accuracy of embodiments of the method. In various examples, an average structure or structural information/data of RNA obtained from single cells (pseudobulk) and that obtained from 10 cells, 100 cells or millions of cells (bulk) has a Pearson correlation coefficient of at least about 0.5, at least about 0.55, at least about 0.6 or at least about 0.65. In one example, an average structure or structural information/data of RNA obtained from single cells (pseudobulk) and that obtained from millions of cells (bulk) has a Pearson correlation coefficient of at least about 0.65. In various embodiments, the method comprises one or more of the following steps: contacting/treating the RNA molecule(s) or cell with an agent capable of modifying the RNA molecule(s) or a modifying agent to obtain modified RNA molecule(s); reverse transcribing the modified RNA molecule(s); sequencing the product obtained from the preceding step to generate sequencing reads; and analysing the sequencing reads to determine the structure of the RNA molecule(s).

In various embodiments, there is provided a method of determining a structure of RNA molecules, the method comprising: contacting the RNA molecules with a modifying agent to obtain modified RNA molecules; reverse transcribing the modified RNA molecules; sequencing the product obtained from the preceding step to generate sequencing reads; and analysing the sequencing reads to determine the structure of the RNA molecules. In various embodiments, the RNA molecules consist of RNA molecules of a single cell or an individual cell. In various embodiments, the RNA molecules are derived from a single cell/individual cell or no more than one cell. In various embodiments therefore, the method is a method of determining a structure of one or more RNA molecules in a single cell.

Nucleic acid structure may be divided into four different levels: primary, secondary, tertiary, and quaternary. In various embodiments, the method may provide information on one or more of these structure levels.

The putative structure of an RNA may be deduced by probing the conformation/folding of the RNA, e.g., with chemical probes. Examples of probes that may be used include, but are not limited to, dimethyl sulfate (DMS), 2 ethyl-3-(3-dimethylaminopropyl)carbodiimide (EDC), N-cyclohexyl-N′-(2-morpholinoethyl)carbodiimide metho-p-toluenesulfonate (CMCT), 3-Ethoxy-α-ketobutyraldehyde (kethoxal), hydroxyl radical, N-methyl-nitroisatoic anhydride (NMIA), 1-methyl-7-nitroisatoic anhydride (1 M7), benzoyl cyanide (BzCN), 1-methyl-6-nitroisatoic anhydride (1 M6), 2-methylnicotinic acid imidazolide (NAI), 2-methyl-3-furoic acid imidazolide (FAI), 2-methylnicotinic acid imidazolide-azide (NAI-N3) and N-propanone isatoic anhydride (NPIA), and derivatives and the like thereof. The probes may be nucleobase-specific probes, such as DMS which can probe adenine (A) and cytosine (C) bases, and EDC, which can probe guanine (G) and uracil (U) bases, or they may be SHAPE (selective 2′-hydroxyl acylation analyzed by primer extension) probes, which create adducts at the 2′-hydroxyl (2′OH) position on the RNA backbone of flexible ribonucleotides with relatively little dependence on nucleotide identity. For example, shape probes may acylate the 2′-OH side chain of a nucleotide (for example A, U, C, or G) that is located at a single strand area. Without being bound by theory, it is believed that a nucleophilic reactivity of ribose 2′-hydroxyl in RNA is higher at conformationally flexible positions and lower or unreactive at nucleotides constrained by base pairing. The propensity of a base to be modified by e.g., a SHAPE reagent at a 2′-hydroxyl position may therefore provide a measure or an indication of the single-strandedness of the base. Bases which are constrained (usually by base-pairing) are less likely be modified than bases which are unpaired.

In various embodiments, the agent for modifying the RNA molecule(s) or the modifying agent is capable of detecting and/or modifying single-stranded bases along an RNA. In various embodiments, the agent for modifying the RNA molecule(s) or the modifying agent preferentially modifies RNA at structurally flexible regions. In various embodiments, the agent preferentially modifies RNA at single-stranded or unpaired regions. In various embodiments, the agent preferentially modifies single-stranded RNA bases or unpaired RNA bases. In various embodiments, the agent preferentially modifies the 2′-hydroxyl of single-stranded RNA bases or unpaired RNA bases e.g., by introducing an adduct at the 2′-hydroxyl positions. In various embodiments, the agent preferentially acylates the 2′-hydroxyl of single-stranded RNA bases or unpaired RNA bases. In various embodiments, the agent comprises a hydroxyl-selective agent. In various embodiments, the agent comprises a hydroxyl-selective electrophilic agent. In various embodiments, the agent comprises a SHAPE reagent. In some embodiments, where the method is carried out on a cell-free RNA, the SHAPE reagent may comprise NMIA, 1M7, BzCN, 1M6, derivatives thereof and/or the like. In various embodiments, where the method is carried out on RNA contained within a cell (e.g., in intracellular probing), the SHAPE reagent may comprise DMS, NAI, NAI-N3, derivatives thereof and/or the like.

In various embodiments, the agent is capable of modifying at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 100% of the single-stranded or unpaired bases/regions in one or more RNA molecules, e.g., in one or more RNA molecules in a single cell. In various embodiments, the agent is capable of modifying substantially all of the single-stranded or unpaired bases/regions in one or more RNA molecules, e.g., in one or more RNA molecules in a single cell. In various embodiments, the agent may produce about 0.3%, about 0.5%, about 1%, about 5%, about 10%, about 20%, about 30%, about 40%, about 50%, about 60%, about 70%, about 80%, about 90% or about 100% more modifications in one or more RNA molecules than a control (such as a DMSO control). In some examples, the agent may produce about 0.5% to about 100% more modifications in one or more RNA molecules than a control. In various embodiments, the agent may produce about 0.3%, about 0.5%, about 1%, about 5%, about 10%, about 20%, about 30%, about 40%, about 50%, about 60%, about 70%, about 80%, about 90% or about 100% more modifications in one or more RNA molecules than DMS. In some examples, the agent may produce about 0.5% to about 100% more modifications than DMS at same/similar concentrations/amounts. In some examples, the agent may produce more than about 1.5 times, more than about 1.6 times, more than about 1.7 times, more than about 1.8 times, more than about 1.9 times, more than about 2 times, more than about 2.1 times, more than about 2.2 times, more than about 2.3 times, more than about 2.4 times, more than about 2.5 times, more than about 2.6 times, more than about 2.7 times, more than about 2.8 times, more than about 2.9 times, more than about 3 times, more than about 3.1 times, more than about 3.2 times, more than about 3.3 times, more than about 3.4 times, more than about 3.5 times, more than about 3.6 times, more than about 3.7 times, more than about 3.8 times, more than about 3.9 times, more than about 4 times, more than about 4.1 times, more than about 4.2 times, more than about 4.3 times, more than about 4.4 times, more than about 4.5 times, more than about 4.6 times, more than about 4.7 times, more than about 4.8 times, more than about 4.9 times, more than about 5 times, more than about 5.1 times, more than about 5.2 times, more than about 5.3 times, more than about 5.4 times, more than about 5.5 times, more than about 5.6 times, more than about 5.7 times, more than about 5.8 times, more than about 5.9 times, more than about 6 times, more than about 6.1 times or more than about 6.2 times more modifications or more average reactivity at each base than DMS at same/similar concentrations/amounts. In some examples, the agent may produce more than about 1.5 times, more than about 1.6 times, more than about 1.7 times, more than about 1.8 times, more than about 1.9 times, more than about 2 times, more than about 2.1 times, more than about 2.2 times, more than about 2.3 times, more than about 2.4 times, more than about 2.5 times, more than about 2.6 times or more than about 2.7 times more modifications or more average reactivity at each base than NAI at same/similar concentrations/amounts.

In various embodiments, the agent is capable of crossing cell membranes. In various embodiments, the agent has high cell permeability. In various embodiments, the agent is capable of entering a cell nucleus. In various embodiments, the method does not comprise a step of isolating RNA from cell(s). For example, RNA need not be isolated from cell(s) prior to the contacting step. In various embodiments, the method does not comprise a step of lysing cell(s) prior to the contacting step.

In various embodiments, the agent has low toxicity or is non-toxic to cells. In various embodiments, the agent is less toxic than DMS at same/similar concentrations.

In various embodiments, the agent comprises NAI or derivatives (e.g., NAI-N3) thereof. In various embodiments, the agent comprises NAI-N3 or derivatives thereof. In various embodiments, a derivative of a compound is structurally related to the compound. For example, the derivative may share a common structural feature, fundamental structure and/or underlying chemical basis with the compound. A derivative is not limited to one produced or obtained from the compound although it may be one produced or obtained from the compound. In some embodiments, the derivative is derivable, at least theoretically, from the compound through modification of the compound. In some embodiments, a derivative of a compound shares or at least retains to a certain extent a function, chemical property, biological property, chemical activity and/or biological activity associated with the compound. A skilled person will be able to identify, on a case-by-case basis and upon reading of the disclosure, the common structural feature, fundamental structure and/or underlying chemical basis of the compound that have to be maintained in the derivative to retain the function, chemical property, biological property, chemical activity, and/or biological activity. A skilled person will also be able to identify assays that can prove the retention of the function, chemical property, biological property, chemical activity, and/or biological activity.

In various embodiments, the agent is provided at a concentration/amount of about 25 mM to about 50 mM per single cell. In some examples, where the agent comprises NAI-N3, the concentration/amount of the agent is about 10 mM to about 80 mM, about 15 mM to about 70 mM, about 20 mM to about 60 mM, or about 25 mM to about 50 mM. In some examples, the agent is provided at a concentration/amount of about 10 mM to about 80 mM, about 15 mM to about 70 mM, about 20 mM to about 60 mM, or about 25 mM to about 50 mM when the method is performed to obtain a single cell RNA structure library. It will be appreciated that other suitable concentrations/amounts may also be used so long as they allow the method to be carried out. The methods for determining the suitable concentrations/amounts are within the purview of a person skilled in the art.

In various examples, the combination of a modifying agent that is in accordance with the embodiments described herein together with a reverse transcription step that is in accordance with the embodiments described herein is shown to advantageously increase modification and/or mutation rates in single-stranded/unpaired bases in RNA. In various examples, the combination increases the modification and/or mutation rates to at least about 5%, at least about 6%, at least about 7%, at least about 8%, at least about 9%, at least about 10%, at least about 11%, at least about 12%, at least about 13%, at least about 14% or at least about 15% in single-stranded/unpaired bases/areas/regions. In various examples, the combination increases the modification and/or mutation rates (or signals or signal-to-noise ratio), optionally the average modification and/or mutation rates (or the average signals or signal-to-noise ratio) by more time 1 time, more than 1.05 times, more than 1.1 times, more than 1.2 times, more than 1.3 times, more than 1.4 times, more than 1.5 times, more than 1.6 times, more than 1.7 times, more than 1.8 times, more than 1.9 times, more than 2 times, more than 2.5 times, more than 3 times, more than 3.5 times, more times 4 times, more than 4.5 times, more than 5 times, more than 5.5 times, more than 6 times, more than 6.5 times, more than 7 times, more than 7.5 times, more than 8 times, more than 8.5 times, more than 9 times, more than 9.5 times, more than 10 times, more than 10.5 times or more than 11 times as compared to a comparative combination in single-stranded/unpaired bases/areas/regions. In various examples, the combination increases the modification and/or mutation rates (or signals or signal-to-noise ratio), optionally the average modification and/or mutation rates (or the average signals or signal-to-noise ratio) in RNA molecule(s) by more time 1 time, more than 1.05 times, more than 1.1 times, more than 1.2 times, more than 1.3 times, more than 1.4 times, more than 1.5 times, more than 1.6 times, more than 1.7 times, more than 1.8 times, more than 1.9 times, more than 2 times, more than 2.5 times, more than 3 times, more than 3.5 times, more times 4 times, more than 4.5 times, more than 5 times, more than 5.5 times, more than 6 times, more than 6.5 times or more than 7 times as compared to a comparative combination. In some examples, the comparative combination may be one of DMS or NAI with one of thermostable group II intron reverse transcriptase (TGIRT) or Invitrogen SuperScript II (SSII) reverse transcriptase. Without being bound by theory, it is believed that the combination not only increases modification of single-stranded/unpaired bases in RNA, but it also increases/favours/promotes RT read-through or jump-through on modified RNA bases, resulting in the recording of an increased number of modification sites as mutations in the reverse-transcribed product (e.g., the cDNA). Whereas a low mutation rate in RNA may require a large number of reads to be obtained for accurate structure determination, a higher mutation rate advantageously allows for accurate structure determination to be achieved with fewer reads. Hence, embodiments of the method may accurately determine a structure or a structural information of RNA at single-cell resolution.

In various embodiments, the reverse transcribing step is carried out in a manganese-containing medium/buffer/solution (e.g., a manganese ion/salt-containing medium/buffer/solution such as a Mn²⁺-containing medium/buffer/solution). In some embodiments, the manganese-containing medium/buffer/solution is substantially free of one or more of other metals (including metals in ionic forms) such as magnesium (e.g., substantially free of magnesium ions/salts such as Mg²⁺), copper (e.g., substantially free of copper ions/salts such as Cu²⁺), cobalt (e.g., substantially free of cobalt ions/salts such as Co²⁺), nickel (e.g., substantially free of nickel ions/salts such as Ni²⁺), lead (e.g., substantially free of lead ions/salts such as Pb²⁺), and/or potassium (e.g., substantially free of potassium ions/salt such as K⁺). In some embodiments, the amount of the one or more of other metals (including metals in ionic forms) in the manganese-containing medium/buffer/solution that is substantially free of one of the one or more other metals is less than about 0.1%, less than about 0.01%, less than 0.001% or less than a detectable amount. In some embodiments, the amount of the one or more other metals (including metals in ionic forms) in the manganese-containing medium/buffer/solution that is substantially free of the of one or more other metals (including metals in ionic forms) is about 0%. The manganese-containing medium/buffer/solution may increase/favour/promote RT read-through or jump-through on modified RNA bases/nucleotides, resulting in mutations being generated at the modified RNA bases/nucleotides. In various examples, a manganese-containing medium/buffer/solution (e.g., a Mn²⁺-containing medium/buffer/solution) more effectively increased/favoured/promoted RT read-through or jump-through on modified RNA bases/nucleotides as compared to a medium/buffer/solution containing magnesium, copper, cobalt, nickel, or lead (e.g., a medium/buffer/solution containing Mg²⁺, Cu²⁺, Co²⁺, Ni²⁺ or Pb²⁺). In various embodiments, the medium/buffer/solution comprises Mn²⁺ as the only divalent metal ion. In various embodiments, the medium/buffer/solution is substantially free of divalent metal ions other than Mn²⁺. In various embodiments, the medium/buffer/solution is substantially free of a divalent metal ion selected from the group consisting of: Mg²⁺, Cu²⁺, Co²⁺, Ni²⁺, Pb²⁺ and combinations thereof. In various embodiments, the medium/buffer/solution comprises a divalent metal ion consisting of Mn²⁺.

In various embodiments, the modifying agent comprises 2-methylnicotinic acid imidazolide (NAI) or derivatives thereof and the reverse transcription step is carried out in a manganese-containing medium/buffer/solution. In various embodiments, the reverse transcribing step is carried out using a reverse transcriptase that is compatible with manganese (e.g., manganese ion/salt such as Mn²⁺). In various embodiments, the reverse transcribing step is carried out using a reverse transcriptase that uses Mn²⁺ as a cofactor. In various embodiments, the reverse transcribing step is carried out using Moloney murine leukemia virus (MMLV) reverse transcriptase. Advantageously, MMLV reverse transcriptase is found to produce more mutations than other reverse transcriptase such as group II intron reverse transcriptase (e.g., TGIRT).

In some embodiments, the MMLV reverse transcriptase comprises a genetically modified/engineered MMLV reverse transcriptase, for example, a genetically modified/engineered MMLV reverse transcriptase with reduced RNase H activity, increased thermal stability and/or enhanced processivity compared to wild-type MMLV reverse transcriptase. Examples of suitable reverse transcriptase include, but are not limited to, members of the Invitrogen SuperScript RT family. In one example, the reverse transcriptase comprises Invitrogen SuperScript II Reverse Transcriptase (SSII RT). In one example, Invitrogen SuperScript II Reverse Transcriptase having an indicated optimal reaction temperature of 42° C. is found to yield better results than Invitrogen SuperScript III Reverse Transcriptase (SSIII RT) having an indicated optimal reaction temperature of 50° C.

In various embodiments, the reverse transcribing step is carried out (or the RT reaction occurs) at a temperature of less than about 50° C., less than about 49° C., less than about 48° C., less than about 47° C., less than about 46° C., less than about 45° C., less than about 44° C. or less than about 43° C. In various embodiments, the reverse transcribing step is carried out (or the RT reaction occurs) at a temperature of no more than about 42° C., no more than about 43° C., no more than about 44° C., no more than about 45° C., no more than about 46° C., no more than about 47° C., no more than about 48° C. or no more than about 49° C. Although the secondary structures of RNA are generally better resolved at higher temperatures, the inventors have surprisingly found out that reverse transcription at a temperature of below 50° C. gives a higher yield than reverse transcription at a temperature of 50° C. In one example, reverse transcription using Invitrogen SuperScript II Reverse Transcriptase at 42° C. is found to give a higher yield than the use of Invitrogen SuperScript III Reverse Transcriptase at 50° C.

The reverse transcribing step may be carried out (or RT reaction occurs) for at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours or at least about 16 hours. In various embodiments, the reverse transcription is performed (or RT reaction occurs) for no less than (or about) 4 hours, no less than (or about) 4.5 hours, no less than (or about) 5 hours, no less than (or about) 5.5 hours, no less than (or about) 6 hours, no less than (or about) 6.5 hours, no less than (or about) 7 hours, no less than (or about) 7.5 hours, no less than (or about) 8 hours, no less than (or about) 8.5 hours, no less than (or about) 9 hours, no less than (or about) 9.5 hours, no less than (or about) 10 hours, no less than (or about) 10.5 hours, no less than (or about) 11 hours, no less than (or about) 11.5 hours, no less than (or about) 12 hours, no less than (or about) 12.5 hours, no less than (or about) 13 hours, no less than (or about) 13.5 hours, no less than (or about) 14 hours, no less than (or about) 14.5 hours, no less than (or about) 15 hours, no less than (or about) 15.5 hours, or no less than (or about) 16 hours. In various embodiments, the reverse transcribing step is carried out for a duration of between about 2-16 hours, about 4-16 hours, about 6-16 hours, about 8-16 hours, about 8-14 hours, about 8-12 hours, about 8-10 hours, about 2-8 hours, about 4-8 hours or about 6-8 hours. In various embodiments, the reverse transcribing step is carried out for a duration of about 4-16 hours or 8-16 hours. A longer duration may increase the efficacy of the reverse transcription. In one example, the reverse transcribing step produces more than 10 times as much product when the duration is increased from 4 hours to 8 hours or 16 hours. In one embodiment, the reverse transcribing step is carried out for a duration of more than 1.5 hours. In one embodiment, the reverse transcribing step is carried out for about 8 hours. Advantageously, the efficiency of reverse transcription is found to be the highest when the duration falls within the duration as disclosed herein.

In various embodiments, the method further comprises fragmenting the nucleic acid molecules e.g., the RNA molecules or the cDNA molecules. The fragmentation of a nucleic acid molecule may produce a plurality of nucleic acid molecules of smaller sizes. In various embodiments, the fragmentation produces a plurality of nucleic acid molecules that are at least about 10 bases (or nucleotides), at least about 20 bases, at least about 30 bases, at least about 40 bases, at least about 50 bases, at least about 60 bases, at least about 70 bases, at least about 80 bases, at least about 90 bases, at least about 100 bases, at least about 110 bases, at least about 120 bases, at least about 130 bases, at least about 140 bases, at least about 150 bases, at least about 160 bases, at least about 170 bases, at least about 180 bases, at least about 190 bases or at least about 200 bases in length. In various embodiments, the fragmentation produces a plurality of nucleic acid molecules that are no more than about 5000 bases, no more than about 4000 bases, no more than about 3000 bases, no more than about 2000 bases or no more than about 1000 bases in length. In various embodiments, fragmentation of the nucleic acid molecules does not result in degradation of the nucleic acid molecules.

In various embodiments, fragmenting the nucleic acid molecules comprises fragmenting the modified RNA molecules. In various embodiments therefore, the method further comprises fragmenting the modified RNA molecules prior to the reverse-transcribing step. Nucleic acid molecules may be fragmented by subjecting them to heat treatment. In various embodiments therefore, fragmenting the modified RNA molecules comprises heating the modified RNA molecules. In various embodiments, the modified RNA molecules are heated in the presence of deoxynucleoside triphosphate (dNTP). Advantageously, embodiments of the fragmentation step are capable of producing RNA fragments of desirable and/or uniform/similar sizes. In various embodiments, embodiments of the fragmentation step produce RNA fragments that are from about 200 bases to about 1000 bases, or about 500 to about 800 bases in length. In some examples, the RNA fragments produced by the fragmenting step have a size distribution of about 200 to about 1000 bases based on bioanalyzer analysis.

In various embodiments, the nucleic acid molecules e.g., the modified RNA molecules are heated to at least about 50° C., at least about 60° C., at least about 70° C., at least about 80° C., at least about 90° C. or at least about 95° C. In various embodiments, the modified RNA molecules are heated to about 90° C., about 91° C., about 92° C., about 93° C., about 94° C., about 95° C., about 96° C., about 97° C., about 98° C., about 99° C. or about 100° C.

In various embodiments, the nucleic acid molecules e.g., the modified RNA molecules are heated for at least about 2 minutes, at least about 5 minutes, at least about 8 minutes or at least about 10 minutes.

In various embodiments, the fragmenting step is carried out in a medium that is substantially free of magnesium (e.g., magnesium ions/salts such as Mg²⁺). In various embodiments, the medium is substantially free of manganese (e.g., manganese ions/salt such as Mn²⁺) and/or potassium (e.g., potassium ions/salt such as K⁺). In various embodiments, the fragmenting step is carried out in a denaturing and/or annealing medium/buffer/solution for reverse transcription. In various embodiments, the fragmenting step is carried out in a medium/buffer/solution (such as a denaturing and/or annealing medium/buffer/solution) comprising/consisting of one of more of the following: water (e.g., distilled water), dNTP, RNase inhibitor and primer such as oligo dT primer. In various embodiments, the water comprises RNase-free water or nuclease-free water. In various embodiments, the medium/buffer/solution is substantially free of RNase and/or nuclease.

In reverse transcription, RNA may be treated with a denaturing and/or annealing medium/buffer/solution to denature the RNA and/or to promote annealing of primer to the RNA. In some embodiments, RNA is treated with a denaturing and annealing medium/buffer/solution to denature the RNA and promote annealing of primer to the RNA simultaneously. In some examples, each cell/RNA is heated in a denaturing and annealing buffer at about 95° C., followed by cooling at about 4° C. for about 10 minutes before reverse transcription. The inventors have surprisingly found that simply heating the modified RNA molecules in a denaturing and/or annealing medium/buffer/solution (or a medium/buffer/solution comprising one or more of water, dNTP (e.g. 1 mM dNTP), RNase inhibitor and primer such as oligo dT primer) not only aids in the denaturing and/or the annealing, but is also aids in fragmenting the RNA into similar and desirable sizes (e.g., about 200-1000 bases) and destroying proteins such as RNase enzyme, thus keeping the fragmented RNA from degradation (e.g., for more than 16 hours). Without being bound by theory, it is also believed that the presence of dNTP during heating may assist in fragmenting the RNA to uniform/similar size (e.g., about 200-1000 bases). In various embodiments therefore, the fragmenting step and a denaturing and/or annealing step are carried out simultaneously. In various embodiments, the fragmentating step and a denaturing and/or annealing step are carried out in similar or identical medium/buffer/solution.

In various embodiments, the fragmentating step and reverse transcribing step are carried out in the same vessel. A vessel may be any container, plate (e.g., multiwell plate), tube, and the like, suitable for carrying out the fragmentating step and reverse transcribing step in accordance with the embodiments described herein. In one embodiment, the vessel comprises a PCR tube, a PCR plate or other types of vessel that can fit into a PCR machine. In one example, following the fragmentation step (which may include also a denaturing and/or annealing step), the reagents/materials for reverse transcription (e.g., 10× reverse transcription buffer, MnCl₂, betaine, SSII reverse transcriptase, template switch primer, DTT) are directly added to the fragmentation medium/buffer/solution (which may be a denaturing and/or annealing medium/buffer/solution) for reverse transcription.

In various embodiments, the method does not comprise a step of separating the RNA, isolating the RNA, purifying the RNA, washing the RNA and/or a step of removing one or more components from the medium/buffer/solution between the fragmentating step and the reverse transcribing step.

In various embodiments, the fragmenting step or heating step confers protection from degradation of the fragmented RNA for more than about 2 hours, more than about 4 hours, more than about 6 hours, more than about 8 hours, more than about 10 hours, more than about 12 hours, more than about 14 hours or more than about 16 hours.

Advantageously, the fragmenting step or heating step produces good quality data for subsequent processing (e.g., for subsequent sequencing), thus allowing for the accurate determination of RNA structure.

The method may also comprise a step of lysing the cell and/or isolating the RNA. The step may be performed after the contacting step and/or before the reverse-transcribing step. In some examples, heat treatment is found to result in both lysis of cell/isolation of RNA and fragmentation of RNA. In some embodiments therefore, the step of lysing the cell and/or isolating the RNA is performed simultaneously with the fragmenting step. In various embodiments, the method comprises a single step that results in two or more of the following: lysis of cell, release/isolation of RNA, denaturing of RNA, annealing of primer to RNA, fragmenting of RNA and destruction/denaturation of undesirable proteins such as RNase enzyme.

In some examples, the cell may be provided as dissociated cell suspension. In some examples, the dissociated cell suspension may be treated with the agent to modify the intracellular RNA molecules. In some examples, following treatment with the agent, the dissociated cell suspension may be separated into single cell suspension (e.g., each individual cell is placed into one vessel).

The cell may be a eukaryotic cell or a prokaryotic cell. In some embodiments, the cell comprises a eukaryotic cell. In some embodiments, the cell comprises a plasma membrane. In some embodiments, the cell does not comprise a cell wall. In some examples, the cell may be a yeast cell. In various embodiments, the cell comprises a mammalian cell. In various embodiments, the mammalian cell comprises a human cell. In various embodiments therefore, the RNA molecules comprise RNA molecules obtained/derived/originating from a eukaryotic cell, a prokaryotic cell, a cell comprising a plasma membrane, a cell devoid of a cell wall, a yeast cell, a mammalian cell or a human cell. Examples of such cells may include, but are not limited to, stem cells, human embryonic stem cells, neuronal precursor cells, neuronal progenitor cells, mature neurons, tumor cells, cancer cells, and the like. The cell may be a healthy cell or a diseased cell.

In various embodiments, the method further comprises amplifying the reverse transcribed product prior to the sequencing step. Amplification reactions known in the art may be employed. The amplification reactions may include but are not limited to polymerase chain reaction (PCR), ligase chain reaction (LCR), loop mediated isothermal amplification (LAMP), nucleic acid sequence based amplification (NASBA), self-sustained sequence replication (3SR), rolling circle amplification (RCA) or any other process whereby one or more copies of a particular nucleic acid sequence may be generated from a nucleic acid template sequence.

In various embodiments, the method further comprises purifying the amplicons or the product from amplification. In various embodiments, the method comprises two or more purification steps. In various embodiments, the method comprises purifying the amplicons for at least about two times, at least about three times, at least about four times or at least about five times. In various embodiments, a purification step is repeated at least about two times, at least about three times, at least about four times or at least about five times. In various embodiments, a purification step is performed or repeated until the product shows a 260 nm/280nm absorbance ratio of about 1.8 and/or a 260 nm/230 nm absorbance ratio of from about 2.0 to about 2.2, about 2.0, about 2.1 or about 2.2. In some examples, the quality of a library is significantly improved when the amplicons are purified for at least about two times. The purification step/protocol may be in accordance with methods known in the art. Examples of purification methods that may be used include, but are not limited to, gel purification, affinity purification, and the like. In one example, beads-based purification (such as AMPure™ beads purification) is used.

In various embodiments, the method further comprises a step of adding a barcode/barcode sequence to the reverse transcribed product or the amplicons. In various embodiments, sequencing is performed on the reverse transcribed products or the amplicons comprising barcodes/barcode sequences. In various embodiments, the method further comprises preparing a library of amplicons. In some examples, the preparation of the library may be performed by using library preparation kits known in the art (for example, Nextera DNA Flex Library Prep kit (Illumina)).

The sequencing step may be performed using methods known in the art. Examples of sequencing techniques include next-generation sequencing, nanopore sequencing, amplicon-based sequencing, paired-end sequencing, Sanger sequencing etc. In some embodiments, the sequencing comprises deep sequencing (e.g., deep sequencing on an Illumina platform, Oxford Nanopore Technologies platform or the like). In some examples, deep sequencing is performed such that the sequencing depth at a base (or a nucleotide) is at least about 5×, at least about 10×, at least about 25×, at least about 50×, at least about 75×, at least about 100×, at least about 150×, at least about 200×, at least about 250× or at least about 300×. In some examples, deep sequencing is performed such that the sequencing depth is at least at least about 50×, at least about 100×, at least about 150×, at least about 200×, at least about 250×, at least about 300×. at least about 350×, at least about 400×, at least about 450×, at least about 500×, at least about 550×, at least about 600×, at least about 650×, at least about 700×, at least about 750×or at least about 800× per 10 bases (or per 10 nt). In some embodiments, the sequencing step generates at least about 5 sequencing reads per base, at least about 10 sequencing reads per base, at least about 25 sequencing reads per base, at least about 50 sequencing reads per base, at least about 75 sequencing reads per base, at least about 100 sequencing reads per base, at least about 150 sequencing reads per base, at least about 200 sequencing reads per base, at least about 250 sequencing reads per base or at least about 300 sequencing reads per base. In some embodiments, the sequencing step generates at least about 50 sequencing reads, at least about 100 sequencing reads, at least about 150 sequencing reads, at least about 200 sequencing reads, at least about 250 sequencing reads, at least about 300 sequencing reads. at least about 350 sequencing reads, at least about 400 sequencing reads, at least about 450 sequencing reads, at least about 500 sequencing reads, at least about 550 sequencing reads, at least about 600 sequencing reads, at least about 650 sequencing reads, at least about 700 sequencing reads, at least about 750 sequencing reads or at least about 800 sequencing reads per 10 bases. In various embodiments, the sequencing step generates no more than about 500 sequencing reads per base or no more than about 400 sequencing reads per base. In various embodiments, the sequencing depth is no more than about 500× or no more than about 400× per base. Advantageously, embodiments of the method, which show improved mutation rates, are capable of more accurately determining RNA structures than conventional methods at the same level of sequencing depth or with the same number of sequencing reads.

In various embodiments, the sequencing step generates at least about 25,000, at least about 50,000, at least about 75,000, at least about 1 million, at least about 5 million, at least about 10 million, at least about 15 million or at least about 20 million sequence reads per cell. In various embodiments, more sequence reads generated per cell may give a better determination of RNA structures. In various embodiments, a higher coverage may give a better determination of RNA structures. In one example, about 15 to about 20 million sequence reads per cell are obtained.

In various embodiments, the analysing step comprises identifying/determining mutations/mutation sites/mutation rate from the sequencing reads. The mutations/mutation sites may indicate the location of the modified bases in the RNA molecule(s). In various embodiments, the method may also comprise determining/evaluating the number/fraction of mutations/modifications or a mutation rate at a particular base of the RNA molecule(s). Without wishing to be bound by theory, it is believed that the number/fraction of mutations or a mutation rate at a particular/specific base can be calculated as an approximate for the likelihood of the base being single-stranded at that location, thus providing structural information along the RNA. For example, a higher number/fraction of mutations or a higher mutation rate at a base may indicate that the base is more likely to be single-stranded. For example, a lower number/fraction of mutations or a lower mutation rate at a base may indicate that the base is less likely to be single-stranded. For identification of mutations/mutation sites from the sequencing reads, the sequencing reads may be mapped to a standard reference. In some embodiments, the analysing step may comprise a step of counting/determining the total read number at a base and the number of mismatch (e.g., mutation, insertion or deletion) at the base. The mutation rate may be calculated by evaluating a ratio of the total read number to the mismatch number. In one example, the mutation rate is calculated by dividing the mismatch number by the total read number (i.e., mutation rate =mismatch number/total read number).

Advantageously, embodiments of the method show high reproducibility and/or accuracy.

In various examples, it was observed that the sum of all the reads along any RNA transcript correlates well with its gene expression information (e.g., information on an amount of RNA transcripts of one or more genes) by traditional RNA sequencing methods. Hence, embodiments of the method may allow one to obtain dual gene expression and RNA structure information at the same time e.g., in a single cell. In various embodiments therefore, there is provided a method of simultaneously determining a structure of an RNA molecule of a gene and an expression of the gene, the method comprising: contacting the RNA molecule with a modifying agent to obtain a modified RNA molecule; reverse transcribing the modified RNA molecule; sequencing the product obtained from the preceding step to generate sequencing reads; analysing the sequencing reads to determine the structure of the RNA molecule of the gene; and evaluating the amount of sequencing reads to determine the expression of the gene. The method may further comprise one or more steps or features as described hereinabove.

In various examples, it was also observed that RNA structure/RNA structural information obtained from embodiments of the method could distinguish between cellular populations, even when the associated gene expression is unchanged/similar in these cellular populations. Thus, embodiments of the method may be used as a biomarker for distinguishing between cell types, including cell types showing similar gene expression profiles and/or transcript/transcriptome profiles. Embodiments of the method may allow for the identification of unique cellular populations in biological systems. In one example, embodiments of the method were able to distinguish between human embryonic stem cells, neuronal precursor cells, neuronal progenitor cells and mature neurons.

In various embodiments therefore, there is provided a method of classifying/sorting/separating/grouping cells into one or more cell populations, the method comprising: determining the structure of RNA molecules in each cell according to embodiments of the method as described herein; and classifying/sorting/separating/grouping the cells into one or more cell populations based on similarity in the structure of their RNA molecules. Cells that are more similar in their RNA structure(s) may be grouped together in a population, while cells that are dissimilar in their RNA structure(s) may be grouped in different populations. In various embodiments, the method is a method of classifying/sorting/separating/grouping cells into different stages of development. In various embodiments, the method is a method of classifying/sorting/separating/grouping cells into different cell types.

Embodiments of the method advantageously provide information on RNA structure at the single cell level, which was previously inaccessible. Embodiments of the method may therefore be harnessed for characterizing a cell/cell population. Characterizing a cell/cell population may comprise identifying a nature and/or a property associated with the cell/cell population. For example, characterising the cell/cell population comprises determining a cell type (including a subtype), a subpopulation (e.g., a functional subpopulation), an expression profile, an RNA structure profile (e.g., for one or more transcripts or transcriptome-wide), a phase, a stage (e.g., a developmental stage) and/or other properties associated with the cell/cell population. In various embodiments therefore, there is provided a method of characterising a cell, the method comprising determining the structure of RNA molecules in the cell according to embodiments of the method as described herein. In various embodiments, there is provided a method of determining the cell type of a cell, the method comprising: determining the structure of RNA molecules in the cell according to embodiments of the method as described herein; and determining the cell type of the cell based on the structure of the RNA molecules. For example, the determined structure of the RNA molecules of the cell may be compared to the pre-determined RNA structure(s) of one or more reference cell(s) of a known cell type(s). If the determined structure of the RNA molecules of the cell is similar/highly similar/identical to a pre-determined RNA structure of a reference cell of a known cell type, the cell may be identified as being of the same type as the reference cell of a known cell type.

In various embodiments, the characterizing/classifying/sorting/separating/grouping may take into account/is based on a gene expression of the cell in addition to its RNA structure/structural information. In various embodiments, the characterizing/classifying/sorting/separating/grouping may take into account/is based on an expression and/or RNA structure/structural information associated with or of a single gene.

In various embodiments, there is provided a kit for determining a structure of RNA molecules, e.g., according to embodiments of the method as described herein, the kit comprising one of more of the following: an agent/a modifying agent in accordance with the embodiments as described herein, a reverse transcription/fragmentation/ denaturing/annealing/lysing medium/buffer/solution in accordance with the embodiments as described herein, a reverse transcriptase as described herein, dNTPs, a Mn²⁺ source, one or more primers (e.g. primers that are capable of binding/hybridizing to RNA and/or cDNA) and a DNA polymerase.

In various embodiments, the kit comprises 2-methylnicotinic acid imidazolide (NAI) or derivatives thereof for modifying the RNA molecules; a reverse transcription medium comprising manganese; and optionally, a Moloney murine leukemia virus (MMLV) reverse transcriptase, optionally a genetically modified MMLV reverse transcriptase. In various embodiments, the kit comprises a single medium, optionally a single starting medium, for two or more of: reverse transcription of the RNA molecules, denaturing of the RNA molecules, annealing of primer to the RNA molecules for reverse transcription and fragmentation of the RNA molecules, wherein the single medium is substantially free of magnesium (e.g., magnesium ions/salt such as Mg²⁺) and/or wherein the single medium comprises dNTPs.

In various embodiments, there is provided a method, a product or a kit as described herein.

### BRIEF DESCRIPTION OF FIGURES

FIG. 1. (A) Testing of different compounds and conditions with Tetrahymena ribozyme gene to obtain higher signal-to-noise ratio and mutation rates. (B) The published secondary structure of Tetrahymena ribozyme was used as a reference to calculate mutation rate accuracy.

FIG. 2. (A)The workflow of single cell RNA secondary structure determination in accordance with embodiments disclosed herein. (B) The size distribution of the library after step 5.

FIG. 3. Splitting of a single cell lysis to form 2 technical replicates to test the reproducibility of embodiments of the single cell RNA structure determination method.

FIG. 4. An evaluation of the reproducibility of embodiments of the single cell RNA structure determination method by comparing the RNA structures determined for single cell, 10 cells, 100 cells and million cells. (A) A model of single cell, 10 cells, 100 cells and million cells. (B) Correlation analysis of determined RNA structures among single cell (pseudo bulk), 10 cells, 100 cells and bulk. (C) The RNA structural signal at each base of ribosomal protein S27 (RPS27) and YY1-associated myogenesis RNA 1 (Yam1) in single cell, 10 cells, 100 cells and million cells are shown as examples.

FIG. 5. The correlation between the expression level of genes calculated from embodiments of the single cell RNA structure determination method using NAI-N3 and the expression level of genes calculated from a previously described single cell RNA seq method using DMSO. Each dot represents a gene.

FIG. 6. An evaluation of the accuracy of embodiments of the single cell RNA structure determination method using 18S ribosomal RNA (rRNA) of cells at different stages of neuronal development as a benchmark. (A) A neurogenesis model from human embryonic stem cells (hESCs) at day 0 (D0) to neuronal precursors at day 7 (D7) to early neurons at day 8 (D8) to neurons at day 14 (D14). (B) The correlation of the determined RNA structure signals of 18S rRNA between bulk and single cell (pseudo bulk) at all the 4 timepoints in neurogenesis. (C) The real RNA structure signal of DO 18S rRNA and D7 18S rRNA at each base in single cell (pseudo bulk) and bulk cells. (D) The real RNA structure signal of D8 18S rRNA and D14 18S rRNA at each base in single cell (pseudo bulk) and bulk cells.

FIG. 7. RNA structure plays roles beyond RNA expression. (A) A neurogenesis model from human embryonic stem cells (hESCs) at day 0 (DO) to neuronal precursors at day 7 (D7) to early neurons at day 8 (D8) to neurons at day 14 (D14). (B) Principal component analysis (PCA) based on RNA structure of the cells at the 4 different development stages and PCA based on expression information of the cells at the 4 different development stages.

FIG. 8. (A) PCA analysis (left) showing that the RNA structure signal of a single gene (NR_024230) could separate different cell types well and heat map (right) showing the RNA structure signal at each base of the gene at different time points. (B) PCA analysis (left) showing that the RNA structure signal of another single gene (TCONS_00029069) could separate different cell types well and heat map (right) showing the RNA structure signal at each base of the gene at different time points.

FIG. 9. (A) Cell-cell RNA structure correlation during neurogenesis process. Structure of human ES cells (DO) are more homogeneous than the structure of cells at other cell stages. (B) The relation of RNA structure heterogeneity with gene expression abundance (top) and structure accessibility (bottom) was evaluated. Structure homogeneity is correlated with structure accessibility but not gene expression abundance.

FIG. 10. RNA structure-based regulation. Changes in the RNA structure in some regions are correlated with the expression RNA-binding protein (RBP), indicating that RBP binding may be responsible for changes in the structure of RNA. Each row in of the heatmap represents a RNA structure signal, while each column represents a cell. Line plot shows the expression of RBP in each single cell as indicated.

FIG. 11. (A) Enrichment analysis were performed in homogeneous regions and heterogeneous regions. A lot of RBPs were enriched in homogeneous regions (light grey dots), but no RBPs were enriched in heterogeneous regions (dark grey dots). (B) The targets of Lin28B, NOLC1 and AQR (overlap with eclip data) were shown to be more homogeneous than those non-target genes. X-axis represents the coefficient of variance. Higher score means the regions are more heterogeneous.

FIG. 12. Comparison of the amount of cDNA products obtained after reverse transcription at different treatment conditions: control, NAI-N3 treatment with use of SuperScript II (SSII) reverse transcriptase and NAI-N3 treatment with use of SuperScript III (SSIII) reverse transcriptase. NAI-N3 treatment with use of SSII gave a higher product yield that NAI-N3 treatment with use of SSIII.

FIG. 13. Comparison of the amount of cDNA products when reverse transcription was allowed to occur for 4 hours, 8 hours and 16 hours for DMSO control and NAI-N3 treatment conditions. Higher product yield was obtained when the reverse transcription reaction occurred for 8 hours and 16 hours.

FIG. 14. Comparison of the quality of library when purification was carried out 1 time and when purification was carried out two times. Quality of the library was improved when purification was carried out two times.

FIG. 15. Comparison of PCR product yield when cell was treated in a lysis buffer comprising 1mM oligodT and 0.2% Triton X to fragment RNA, and when cell was heated in a composition comprising 2.5 mM oligodT and water to fragment RNA. The latter condition was found to increase PCR product yield by 3-4 folds.

### EXAMPLES

Example embodiments of the disclosure will be better understood and readily apparent to one of ordinary skill in the art from the following discussions and if applicable, in conjunction with the figures. It will be appreciated that the example embodiments are illustrative, and that various modifications may be made without deviating from the scope of the invention. Example embodiments are not necessarily mutually exclusive as some may be combined with one or more embodiments to form new exemplary embodiments.

To better identify single cell populations, a single cell RNA structure probing method has been developed to complement structural information with gene expression information in single cells. This method allows RNA structure to be used as a biomarker to identify functional cellular populations that have different structures from other populations that share similar gene expression profiles. The inventors have named this method ‘deciphering identity of single cells operated through structure-“DISCOS”’.

Importantly, in order to detect RNA structures robustly in single cells, the number of mutations generated by a structure probe needs to be high. Different structure probing compounds and different reverse transcription strategies were tested to identify conditions that allowed for the robust identification of single-stranded structure modifications using mutation mapping and deep sequencing. It was observed that technical replicates of DISCOS were highly reproducible, as evident from the high structural correlations in a single cell transcriptome that was split into two technical replicates. High reproducibility between the averaged single cell structure data and millions of cells was also observed, again suggesting that the data was of high quality.

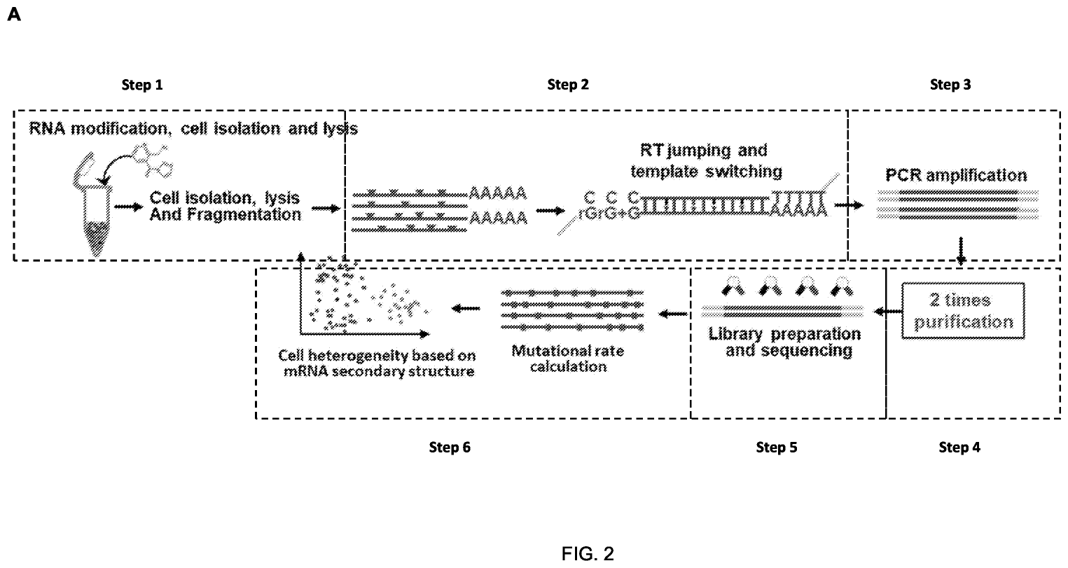

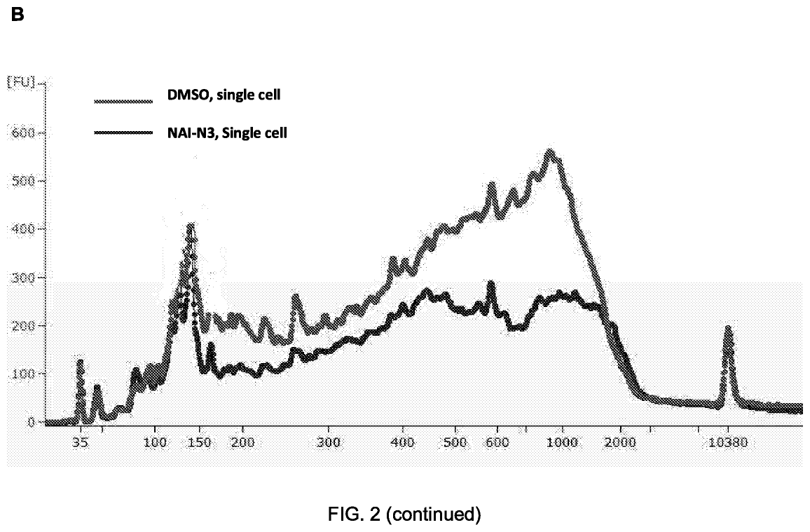

A workflow of the method in accordance with embodiments disclosed herein is shown in FIG. 2A. Briefly, in step 1, a modifying agent is added to a dissociated cell suspension to modify the intracellular RNA molecules. Following treatment with the modifying agent, the dissociated cell suspension may be separated into single cells and then lysed to release the modified RNA molecules from each single cell. The RNA molecules may be fragmented into smaller sizes. In step 2, the modified RNA molecules are reverse transcribed. The reverse transcriptase may “jump” through a modified RNA base and incorporate an erroneous base or a mutation in the cDNA during synthesis. Template switching may also occur with the use of certain reverse transcriptase. In step 3, the cDNA products undergo PCR amplification. The amplicons may be purified in the next step to remove unwanted materials from the amplification process. An at least 2 times purification in step 4 may further improve the quality of the RNA for downstream uses. Next, in step 5, library preparation and sequencing are carried out. The sequence reads obtained are analysed in step 6 to identify positions/regions having a high mutation rate. A high mutation rate at a base/region may indicate that the base/region is single-stranded. By calculating the mutation rate at each position/region therefore, the structure of the RNA molecules may be elucidated. The RNA structure information may be further harnessed for uses such as sorting of cells into different cell types.

### Selection of Compounds and Conditions to Increase Mutation Rates

Reagents that may be used for RNA structure probing include dimethyl sulfate (DMS), N-cyclohexyl-N′-(2-morpholinoethyl)carbodiimide metho-p-toluenesulfonate (CMCT), 3-Ethoxy-α-ketobutyraldehyde (kethoxal), hydroxyl radical, N-methyl-nitroisatoic anhydride (NMIA), 1 -methyl-7-nitroisatoic anhydride (1 M7), benzoyl cyanide (BzCN), 1-methyl-6-nitroisatoic anhydride (1M6), 2-methylnicotinic acid imidazolide (NAI), 2-methyl-3-furoic acid imidazolide (FAI), 2-methylnicotinic acid imidazolide-azide (NAI-N3) and N-propanone isatoic anhydride (NPIA) (see Table 1 below).

To increase the “jump” through rates, numerous different structure probing compounds were tested, as well as numerous different RT enzymes and RT conditions. NMIA, 1M7, BzCN, 1M6 are less cellular permeable and hence are not suitable for in vivo RNA structure probing. DMS, NAI, NAI-N3 are cell permeable compounds. However, DMS only label As and Cs out of 4 bases and can be toxic to cells, resulting in their death and the degradation of RNAs. NAI and NAI-N3 could do structure probing in vivo and result in less cell death at relative low concentration. The inventors compared NAI with NAI-N3 in a single cell RNA structure probing protocol. The results show lower mutation mapping being obtained with the use to NAI as compared to NAI-N3 at similar concentrations. Hence, NAI-N3 was chosen for single cell RNA structure mapping.

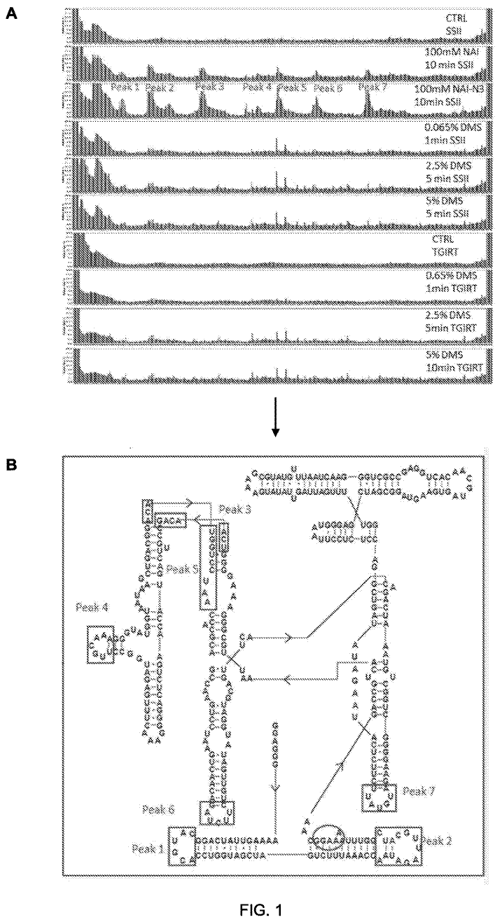

The tests were performed on Tetrahymena ribozyme. The results are shown in FIG. 1, and Tables 1 and 2 below.

TABLE 1
The average reactivity of each base of Tetrahymena
ribozyme at different treatment conditions.

| | Average reactivity of each |
|---|---|
| Treatment condition | base (Treatment-CTRL) (%) |
| 2.5% DMS_TGIRT | 0.3041 |
| 5% DMS_TGIRT | 0.4484 |
| 2.5% DMS_SSII | 0.7506 |
| 5% DMS_SSII | 0.7826 |
| 50 mM NAI_SSII | 0.6992 |
| 50 mM NAIN_SSII | 1.8982 |

TABLE 2
The average mutation rates and signal-to-noise ratios of
Tetrahymena ribozyme at different treatment conditions.

| | | Avg. | Avg. | |
|---|---|---|---|---|
| | | mutation | mutation | |
| | | rate- | rate- | Signal |
| | Avg. | Single | Double | to |
| | mutation | strand | strand | noise |
| Condition | rate | area | area | ratio |
| SSII_control | 0.423% | 0.374% | 0.425% | 88.035% |
| TGIRT_control | 0.347% | 0.345% | 0.353% | 97.827% |
| 2.5% | 0.621% | 0.915% | 0.444% | 205.908% |
| DMS_TGIRT | | | | |
| 5% DMS_TGIRT | 0.769% | 1.197% | 0.535% | 223.838% |
| 2.5% DMS_SSII | 1.251% | 1.451% | 0.754% | 192.472% |
| 5% DMS_SSII | 1.182% | 1.630% | 0.901% | 180.981% |
| 100 mM NAI_SSII | 1.092% | 1.716% | 0.670% | 256.207% |
| 100 mM | 2.304% | 3.860% | 1.221% | 316.163% |
| NAIN3_SSII | | | | |

It was found that by using SHAPE-like compound NAI-N3, and RT enzyme SSII with Mn²⁺, average mutation rate in single-stranded bases could be increased to about 3.86%, with some single-stranded bases recording a mutation rate of more than about 10% (Table 2 and FIG. 1). This represents an at least 10.3 times increase in average mutation rates as compared to the control conditions, an at least 3.2 times increase in average mutation rates as compared to the use of DMS in combination with RT enzyme TGIRT, and an at least 2.3 times increase in average mutation rates as compared to the use of DMS in combination with RT enzyme SSII in single-stranded bases. Both NAI and NAI-N3 result in greater mutation rates as compared to DMS in single-stranded bases.

As the combination of NAI-N3 with SSII generated the highest average mutation rate and signal-to noise ratio, this combination was selected for single cell RNA secondary structure determination.

### Establishing a Suitable RNA Fragmentation Condition Prior to Library Preparation

RNA fragmentation after SHAPE structure probing is important to enable reverse transcription to travel to the end of the RNA fragment to allow PCR amplification. Traditionally, RNA fragmentation conditions involve MgCl₂, KCl in Tris Buffer. However, as the Mg²⁺ in the buffer affects reverse transcription reaction, which uses Mn²⁺, instead, other buffers suitable for fragmentation had to be identified.

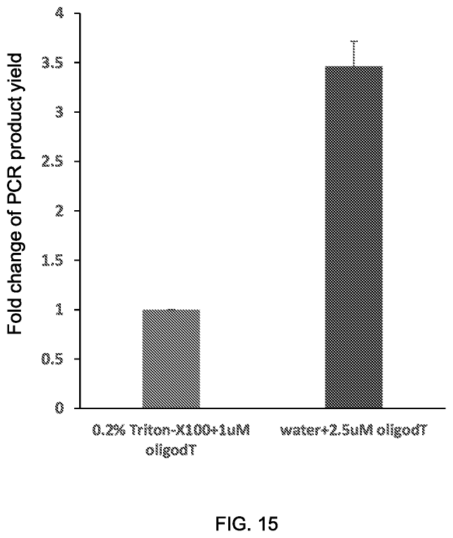

Mn²⁺ and K⁺ were first tested to see whether they could fragment RNAs in water, but they didn't work. The inventors then tested a combination of water and Triton X for fragmentation, but the combination did not work as well. Eventually, it was found out that heating RNA in water and dNTP was able to fragment RNA to a size that was desirable for single cell structure probing. Further, cell lysis, denaturing of RNA, and fragmenting of RNA were achieved in this single step of heating RNA. This step contributes to successful library preparation. Bioanalyzer analysis carried out after library preparation showed that the size distribution was from about 200-1000 bases (see FIG. 2B) As compared to the use of 1 mM oligodT and 0.2% Triton X in a lysis buffer, heating RNA in 2.5 mM oligodT and water was found to increase subsequent PCR amplification yield by 3-4 folds (FIG. 15).

### Increasing the Efficiency of Reverse Transcription

As reverse transcriptase works slower in buffer conditions containing Mn²⁺ as compared to Mg²⁺, the duration of reverse transcriptase was optimised to obtain the most efficient cDNA synthesis. Instead of the usual 1.5 hours for reverse transcription, it was found that the efficiency of reverse transcriptase was the highest when the reaction was allowed occur for about 8 hours. The ratio of the amount of cDNA products for the reaction durations of 4 h:8 h:16 h was 1:11.4:11.5 (NAI-N3) (FIG. 13). In the control condition, the ratio for the reaction durations of 4 h:8 h:16 h was 1:12:8 (DMSO).

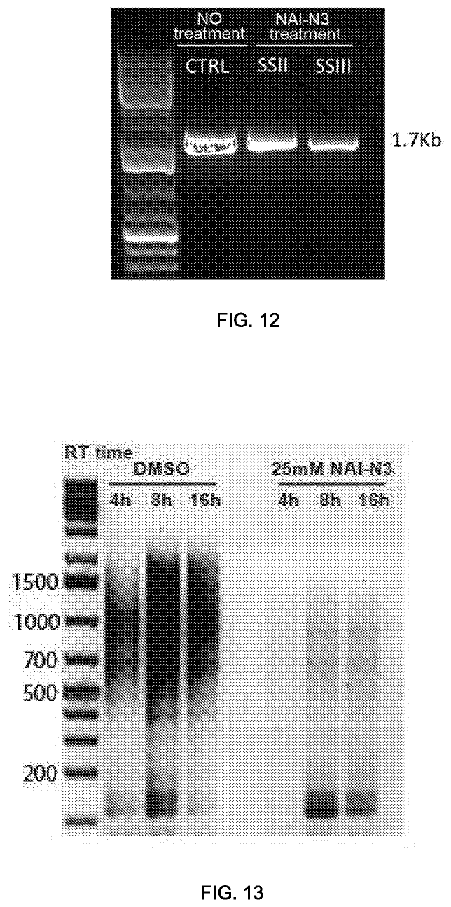

Furthermore, the inventors also found that the use of SuperScript II reverse transcriptase at 42° C. resulted in a higher product yield as compared to SuperScript III reverse transcriptase at 50° C. (FIG. 12).

### Double Purification Improves Quality of Library

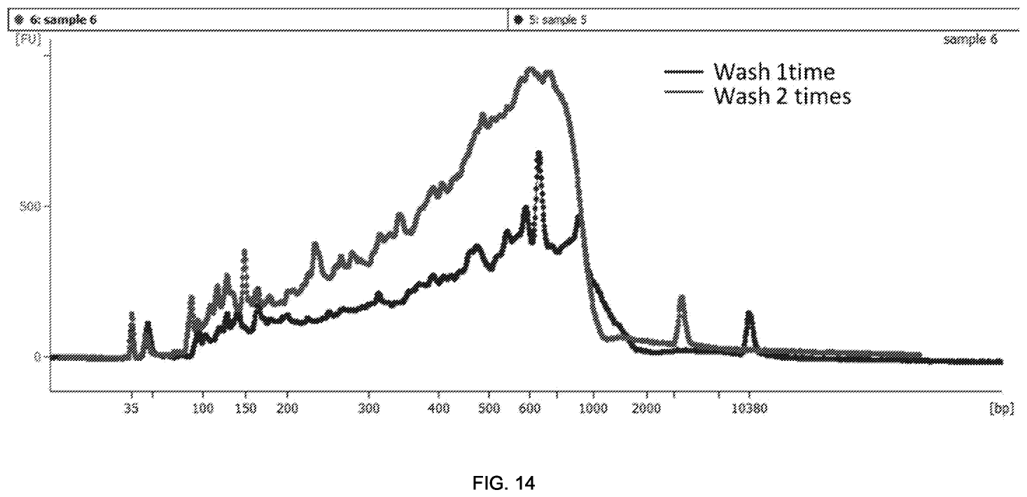

Purification steps after PCR amplification are helpful for obtaining good quality libraries. A double purification, as compared to single purification, resulted in a library showing a higher intensity and less peaks, indicating that additional purification improves the quality of the library (FIG. 14). Double purification was performed before the eventual sequencing.

### DISCOS is Reproducible and Accurate

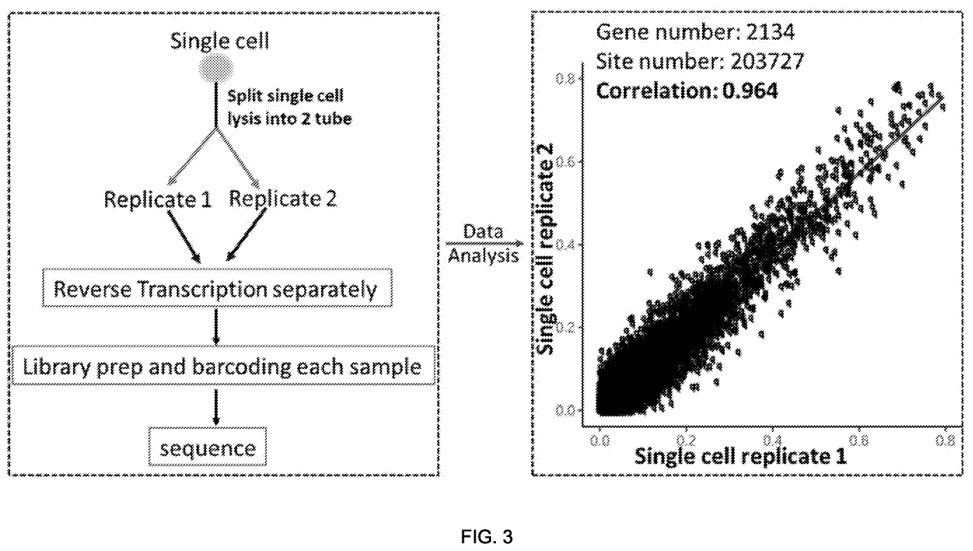

When the transcriptome of a single cell was split into two technical replicates, high structure correlations between the two technical replicates were observed, suggesting the structure probing results are reproducible (FIG. 3).

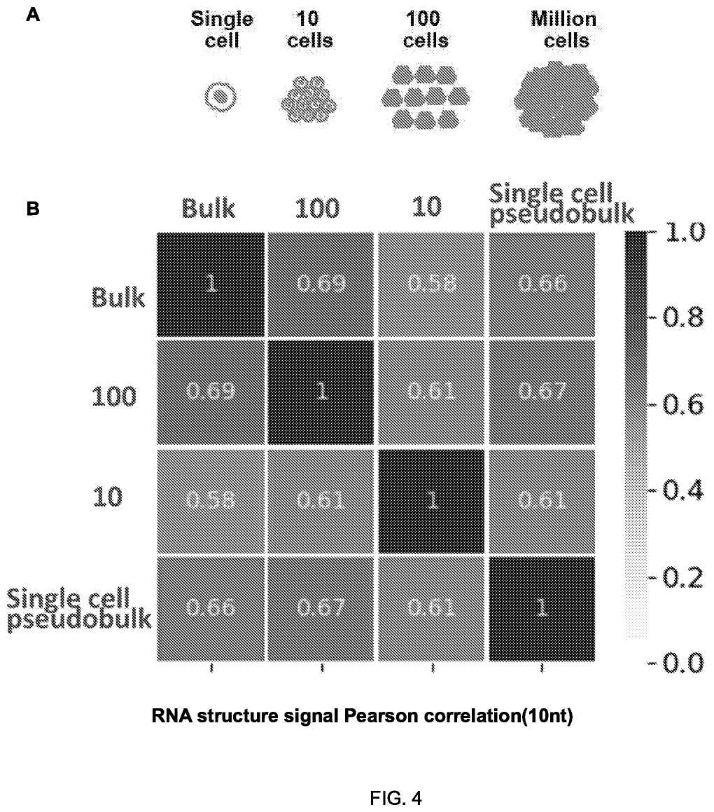

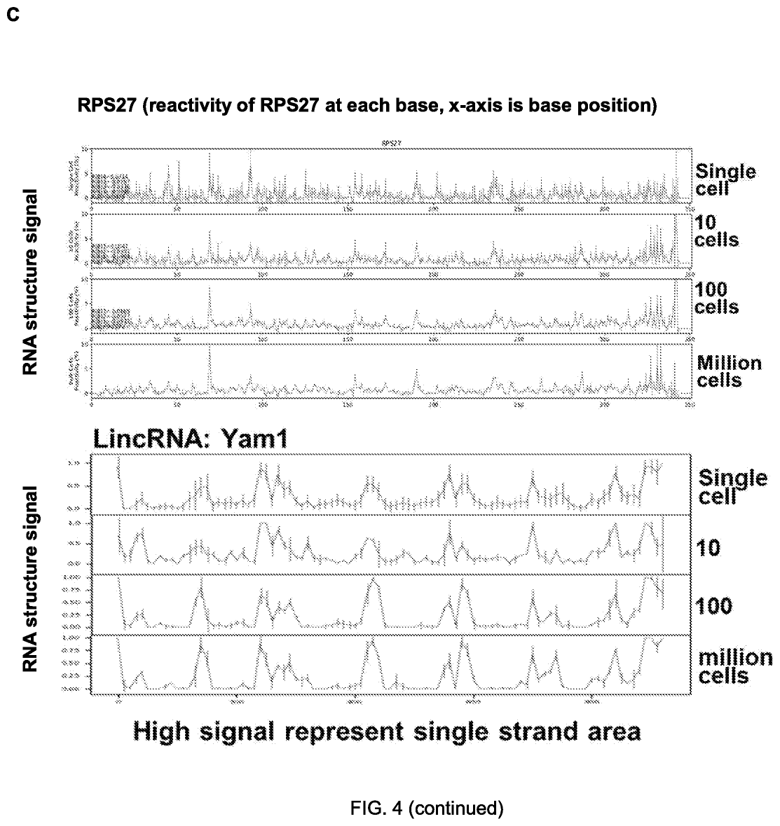

Comparing the average of single cell structure signals to the structure signals from 10 cells, 100 cells and millions of cells also show high correlations, suggesting that the structure probing is accurate (FIG. 4A and FIG. 4B). The RNA structural signal at each base of ribosomal protein S27 (RPS27) and YY1-associated myogenesis RNA 1 (Yam1) in single cell, 10 cells, 100 cells and million cells are shown as examples in FIG. 4C. The method is accurate, reproducible and stable.

### DISCOS Captures RNA Expression Level and RNA Structure Information at the Same Time

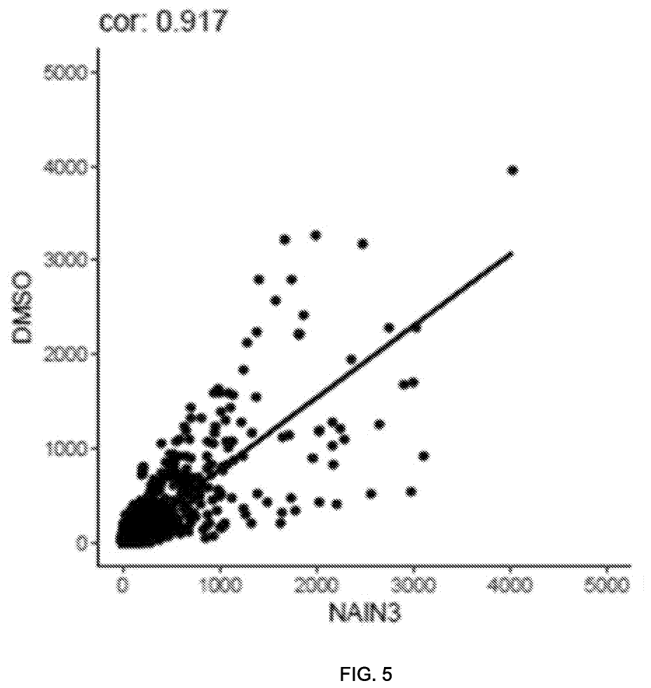

Interestingly, it was also observed that the sum of all the reads along any transcript correlates well with its gene expression information by traditional RNA sequencing (FIG. 5). This suggests that DISCOS can obtain dual gene expression and RNA structure information at the same time in a single cell. This combination of information can enable one to combine structural and gene expression differences between single cells to classify cellular populations better.

### RNA Structure Information from DISCOS could Distinguish between Cellular Populations

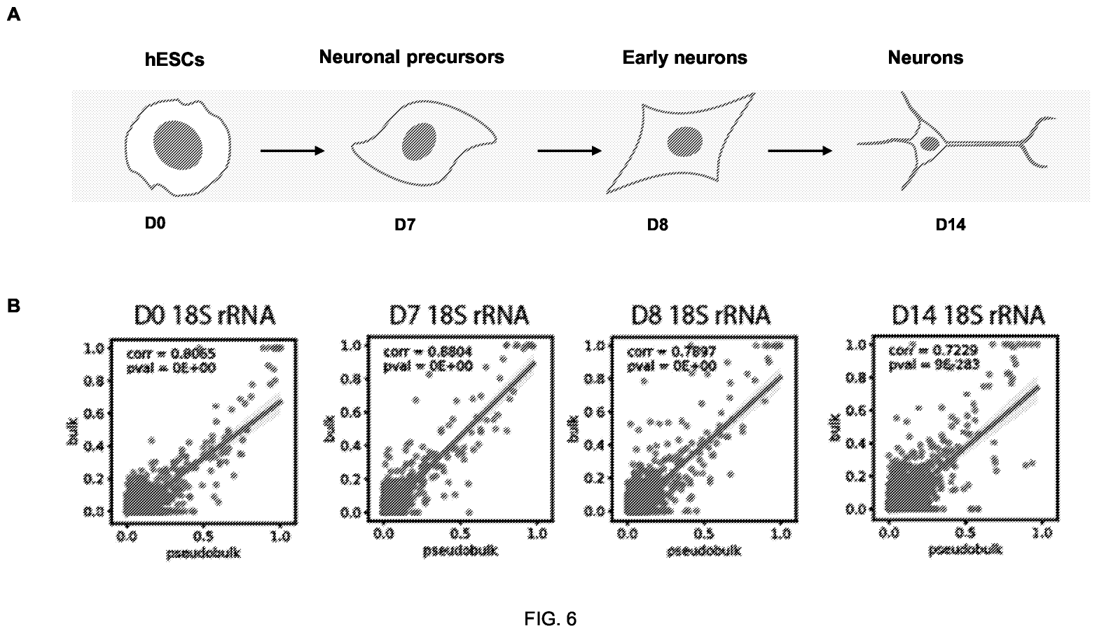

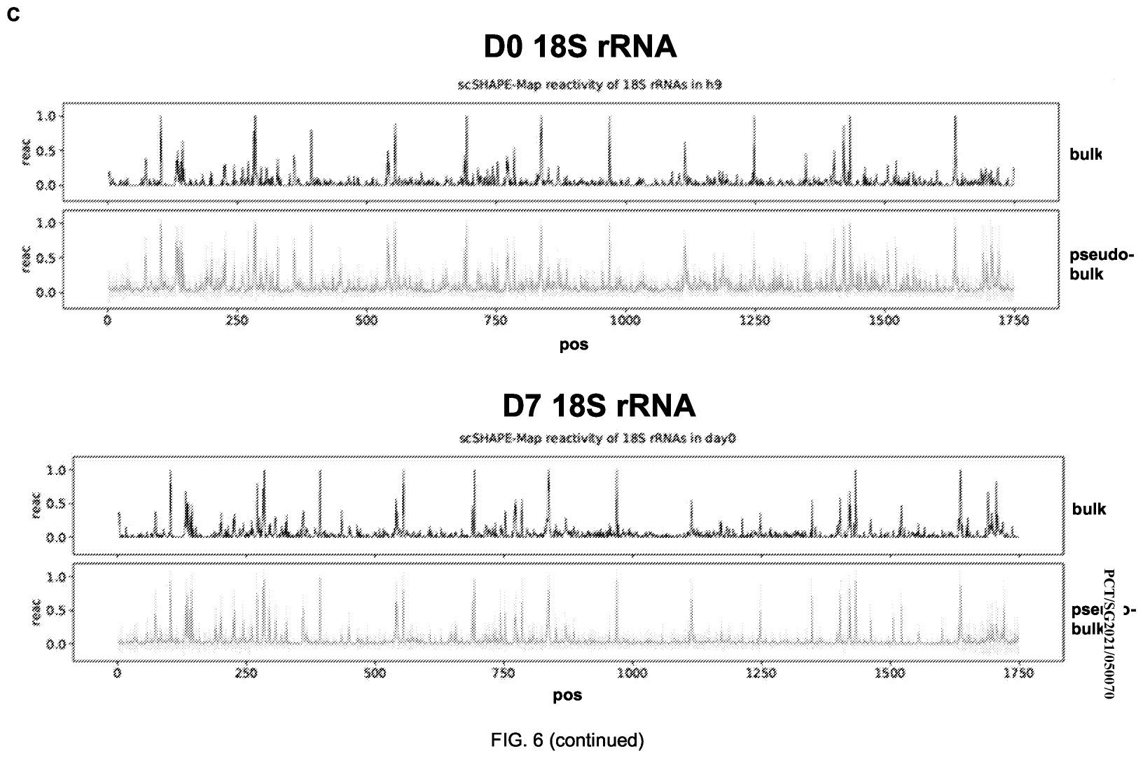

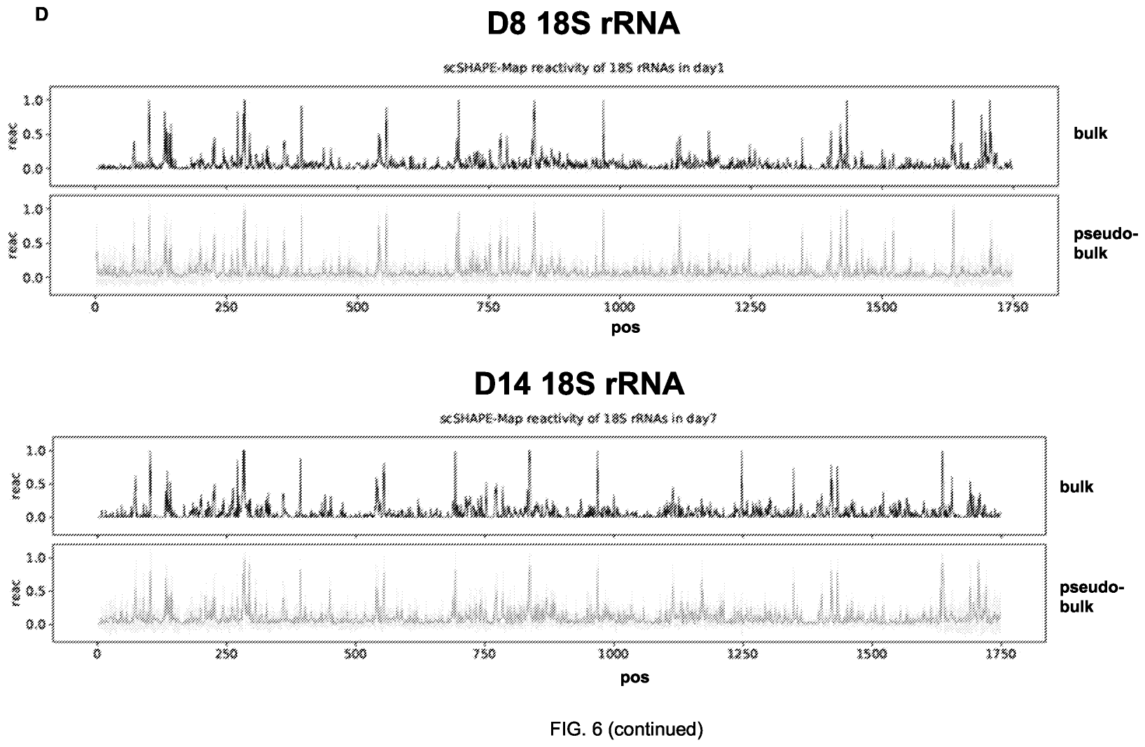

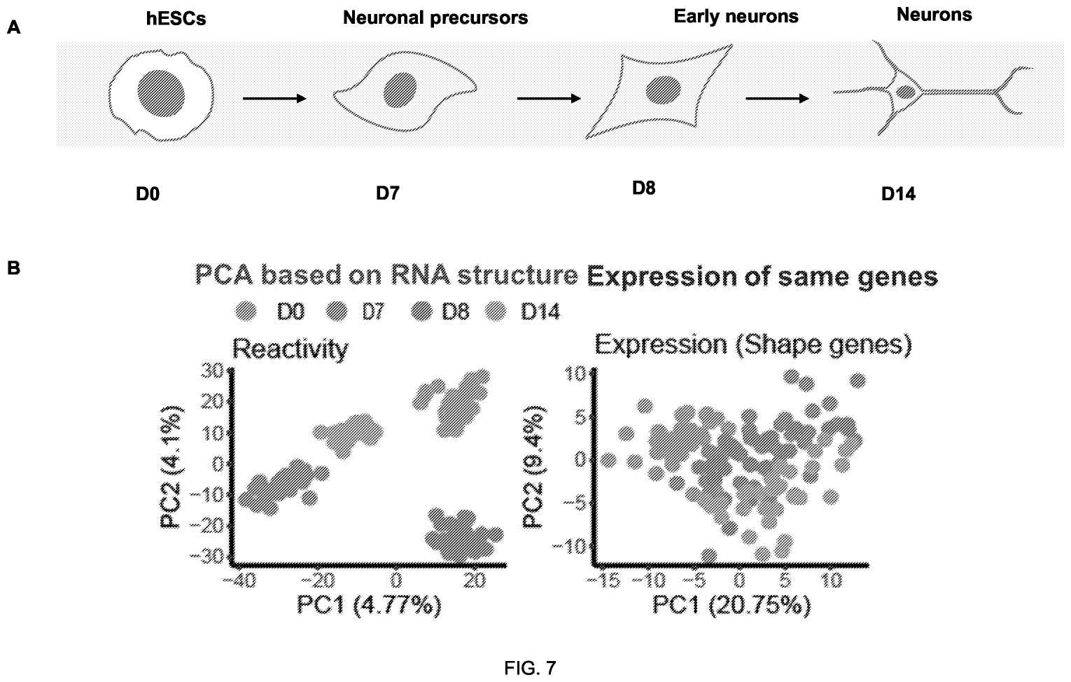

To show that RNA structures are important during development, single cell RNA structure probing was performed in human embryonic stem cells, neuronal precursor cells, neuronal progenitor cells and mature neurons (FIG. 6). It was observed that structural information adds a secondary layer of information on top of gene expression (FIG. 7), as the inventors could distinguish cellular populations based on RNA structure information alone (FIG. 7B left), on transcripts that do not change gene expression (FIG. 7B right). The single cell RNA structure data obtained from DISCOS could cluster cells at different stages of development well, but the expression level of the same gene could not separate the cells at different stages of development. Hence, the single cell RNA structure data obtained from DISCOS is able to cluster cells at different stages of development better than RNA expression level.

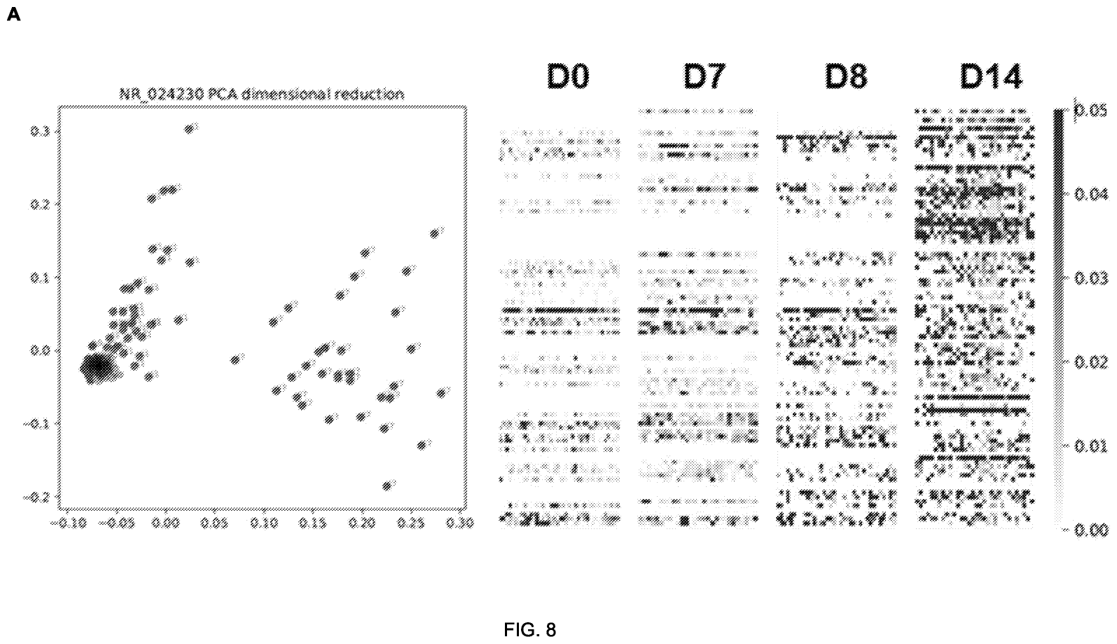

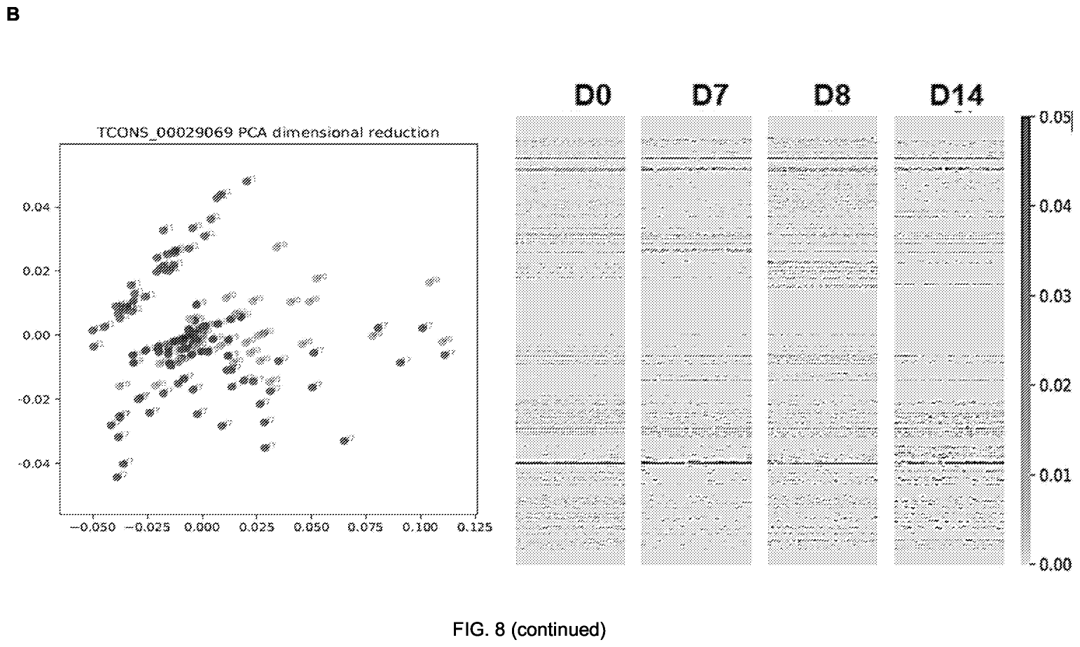

Interestingly, it was found that the structure information of some genes could group individual cells of different cell types together (FIG. 8), thus suggesting that RNA structure could serve as biomarkers for cell types.

### RNA structure Homogeneity is Correlated with Structure Accessibility

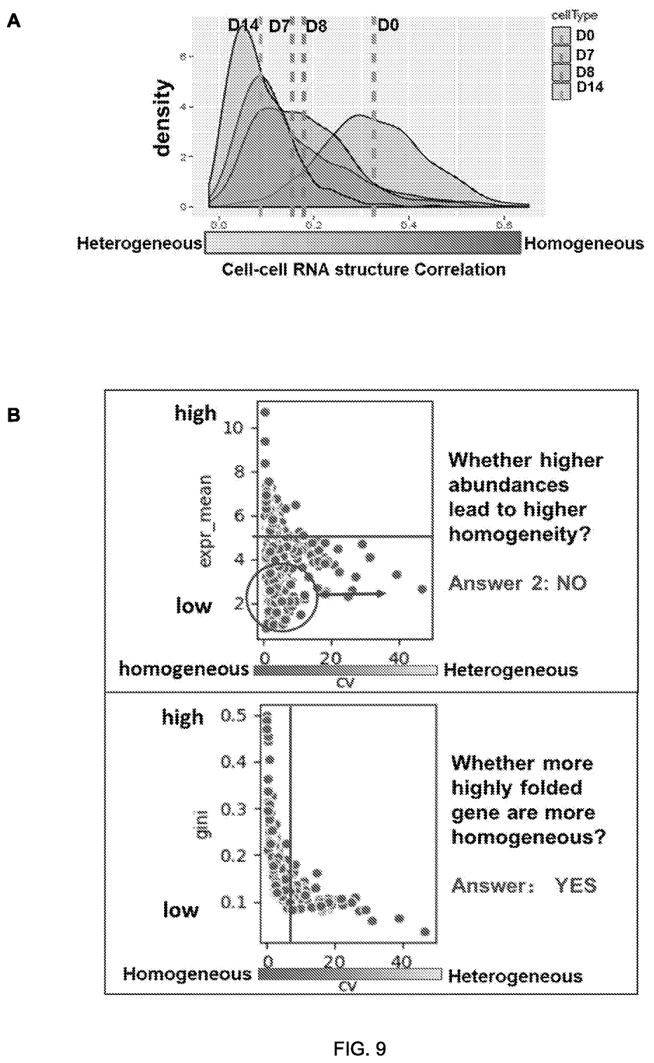

Cell-cell RNA structure correlation was investigated for the cells at the different stages of development. Structure of human ES cells (DO) was found to be more homogeneous than cells at the other developmental stages (FIG. 9A). The relation of RNA structure heterogeneity with gene expression abundance and structure accessibility was also tested. Structure homogeneity was found to be correlated with structure accessibility but not gene expression abundance (FIG. 9B).

### RBP Binding is a Regulator of RNA Structure

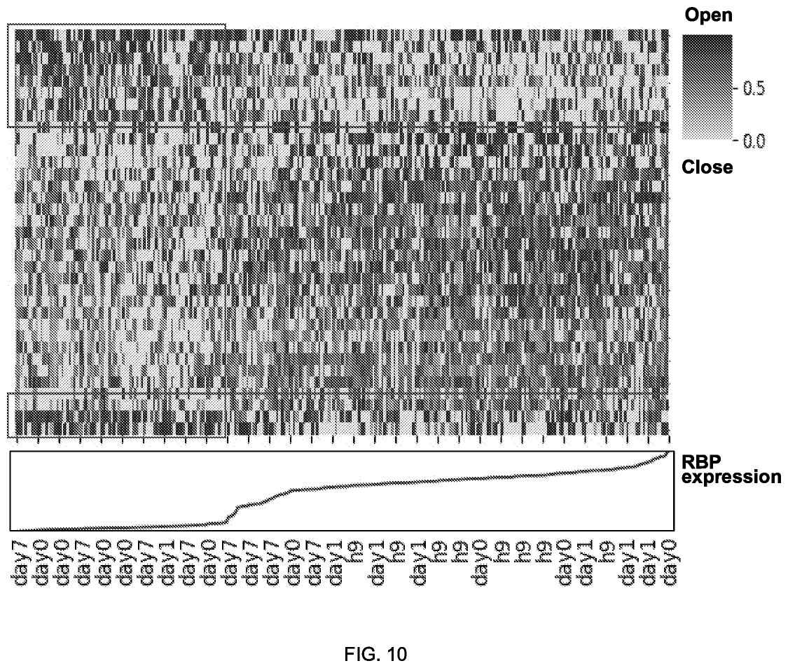

The change in RNA structure at some regions was observed to be correlated with RBP's expression, indicating RBP binding is one reason contributing to structure change (FIG. 10)

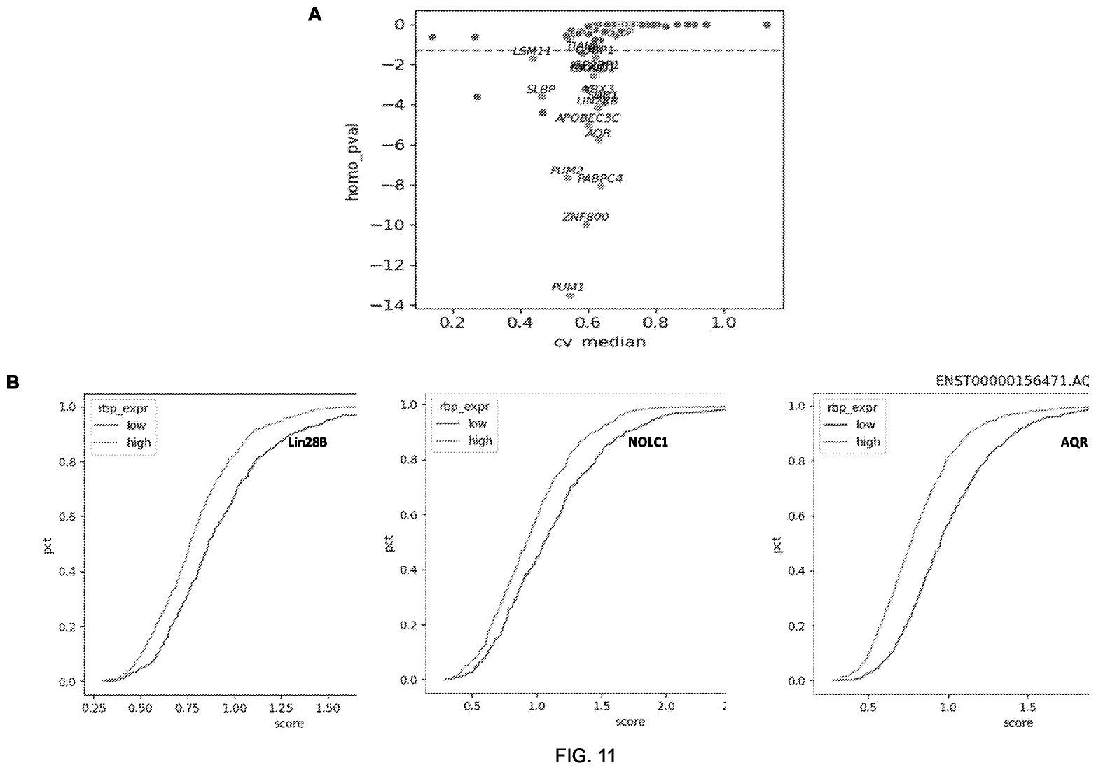

Enrichment analysis was performed in homogeneous regions and heterogeneous regions. A large fraction of RBPs were enriched in homogeneous regions, but no RBP was enriched in the heterogeneous regions (FIG. 11A). The targets of Lin28B, NOLC1 and AQR were shown to be more homogeneous than those non-target genes (FIG. 11B). These show that RBP binding makes the RNA structure of their targets more stable.

### Materials and Methods

### Cell Lysis, Denaturing of RNA, Annealing of Primer to RNA and Fragmenting of RNA

MIX A (see Table 3 below) was prepared and added to a PCR tube containing the single cell. The RNase inhibitor used was Invitrogen™ SUPERase•In™ RNase Inhibitor (20 U/μL) purchased from Thermo Fisher Scientific (Catalog number: AM2694).

TABLE 3
The components of MIX A and their amounts.

| | | Working | Amount |
|---|---|---|---|
| Component | Stock | concentration* | (μl) |
| RNase inhibitor | stock | — | 0.1 |
| oligo dT | 50 μM | 2.5 μM | 0.5 |
| dNTP | 10 mM | 1 mM | 1 |
| single cell solution | — | — | 0.5 |
| water | — | — | 1.9 |

| | |
|---|---|
| Total: | 4 |

*Working concentration here refers to the concentration of each component in a 10 μl reaction mix after a 6 μl RT reaction mix is added in the next step.

The following program was run:

TABLE 4
Program setting out the temperature and duration at each stage.
Program

| | |
|---|---|
| 95° C. | 10 min |
| 4° C. | 10 min |
| 72° C. | 3 min |
| 4° C. | 10 min |
| 4° C. | hold |

### Reverse Transcription

Following fragmentation, Mix B (see Table 5 below) was prepared and added into PCR tube from the previous step to give a 10 μl reaction mix (4 μl Mix A+6 μl Mix B) for reverse transcription. The concentration of the betaine stock solution is 5 M.

TABLE 5
The components of Mix B and their amounts.

| Mix B (reverse | | | Working | | Amount |
|---|---|---|---|---|---|
| transcription) | Stock | | concentration | | (μl) |
| Water | — | | — | | 0.96 |
| First stand buffer | 10X | | 1X | | 1 |
| TSO | 25 | uM | 0.1 | uM | 0.04 |
| RNase inhibitor | 20 | U/ul | 1 | U/ul | 0.5 |
| DTT | 100 | mM | 5 | mM | 0.5 |
| MnCl₂ | 120 | mM | 6 | mM | 0.5 |
| SSII | 200 | U/ul | 10 | U/ul | 0.5 |

| | | | |
|---|---|---|---|
| Betaine | 5X | 1X | 2 |

| | |
|---|---|
| In Total | 6 |

The following program was run:

TABLE 6
Program for reverse transcription
Program

| | |
|---|---|
| 25° C. | 5 min |
| 42° C. | 8 hours |
| 70° C. | 10 min |
| 4° C. | Hold |

### Polymerase Chain Reaction (PCR) Amplification

Following reverse transcription, Mix C (see Table 7 below) was prepared and added into the PCR tube from the previous step to obtain a 25 μl reaction mix for PCR amplification. The high-fidelity PCR mix used was KAPA HiFi HotStart ReadyMix (Catalog number: KK2602).

TABLE 7
The components of Mix C and their amounts.

| Mix C (PCR amplification) | Amount (μl) |
|---|---|
| High-fidelity PCR mix | 12.5 |
| ISPCR primers (10 uM) | 0.25 |
| water | 2.25 |
| In total: | 15 |

The following program was run:

TABLE 8
Program for PCR amplification
Program (24-25 cycles)

| | |
|---|---|
| 98° C. | 3 min |
| 98° C. | 20 sec |
| 67° C. | 15 min |
| 72° C. | 6 min |
| 72° C. | 5 min |
| 4° C. | hold |

### Beads Purification

AMpure beads were used for purification. Briefly, AMpure beads were added to the PCR product (1:1 ratio) and incubated for 8 minutes. The mixture was then placed on the magnetic stand for 5 minutes, after which liquid was removed and the beads were washed using 70% ethanol for 30 seconds. Washing was repeated. Finally, the DNA was eluted from the beads by 10 μl water. The steps above were repeated in a second round of purification.

### Library Preparation and Sequencing

The purified PCR product derived from single cells was used to prepare a library using Illumina Nextera XT DNA Sample Preparation kit (FC-131-1096). Briefly, 0.5 ng of PCR product was treated by tagmentation reaction (2.5 μl Tagment DNA Buffer+1.25 μl Amplification Tagment Mix), at 55° C. for 5 minutes. Unique barcodes were then added for each reaction and PCR was performed following the kit guide. Next, all the PCR products derived from the different single cells were combined and purified using Ampure beads. The sample was then sequenced by highseq4K (pair end 150 bp).

### Analysis

Briefly, the reads were mapped to the longest transcriptome using bowtie2. For the competent regions with enough reads coverage (100 reads per nt/600 reads per 10 nt), the mutants were detected using bam-readcount and the mutant rate were calculated by using an in-house script (https://github.com/genome/bam-readcount.git). Then, any nt/win that was missing in more than 50% cells of each stage was filtered out. Genes that are shorter than 50 nt were also filtered out. The reactivity was calculated by subtracting the mutant rate in DMSO control from the read count mutant rate in NAI-N3. If the subtraction resulted in a negative value, the value was masked as 0. The reactivity was quantile normalized (or not in some cases) and scaled to 0-1.

By calculating the correlation distance of each gene, the RNA structure heterogeneity was measured at the gene level. By using the coefficient of variance of reactivity in each win/nt, RNA structure was measured at win/nt level.

### Simultaneous Determination of Both Expression and RNA Structure of a Gene

After sequencing, the number of reads is counted. The reads number represents the gene expression level. The mutational rate at each base is also calculated. The mutation number is then divided by reads number to thus obtain expression and RNA structure information at the same time.

### Principle Component Analysis for Clustering of Cells

Principle component analysis (PCA) was performed based on RNA structure information or expression information using PCA package in R with parameter (n_components=2).

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of determining a structure of ribonucleic acid (RNA) molecules, the method comprising:
contacting the RNA molecules with a modifying agent to obtain modified RNA molecules;
reverse transcribing the modified RNA molecules;
sequencing the product obtained from the preceding step to generate sequencing reads; and
analysing the sequencing reads to determine the structure of the RNA molecules;
optionally wherein the method further comprises fragmenting the modified RNA molecules prior to the reverse-transcribing step.

2. The method according to claim 1, wherein the modifying agent comprises 2-methylnicotinic acid imidazolide (NAT) or derivatives thereof.

3. The method according to claim 1, wherein the reverse transcribing step is carried out in a manganese-containing medium, optionally wherein the reverse transcribing step is carried out at a temperature of less than 50° C.

4. The method according to claim 1, wherein the modifying agent comprises 2-methylnicotinic acid imidazolide (NAI) or derivatives thereof and wherein the reverse transcription step is carried out in a manganese-containing medium.

5. The method according to claim 1, wherein the reverse transcribing step is carried out using Moloney murine leukemia virus (MMLV) reverse transcriptase, optionally a genetically modified MMLV reverse transcriptase.

6. The method according to claim 1, wherein the reverse transcribing step is carried out for least about 2 hours, optionally at least about 4 hours, further optionally at least about 6 hours, further optionally at least about 8 hours.

7. (canceled)

8. The method according to claim 1, wherein fragmenting the modified RNA molecules comprises heating the modified RNA molecules.

9. (canceled)

10. The method according to claim 1, wherein the fragmenting step is carried out in a medium that is substantially free of magnesium.

11. The method according to claim 1, wherein the fragmenting step is carried out in the same vessel as the reverse transcribing step.

12. The method according to claim 1, wherein the method further comprises amplifying the reverse transcribed product prior to the sequencing step.

13. The method according to claim 1, wherein the method further comprises purifying the amplicons, further optionally purifying the amplicons for at least two times.

14. The method according to claim 1, wherein the RNA molecules consist of RNA molecules of a single cell.

15. A method of simultaneously determining a structure of an RNA molecule of a gene and an expression of the gene, the method comprising:
contacting the RNA molecule with a modifying agent to obtain a modified RNA molecule;
reverse transcribing the modified RNA molecule;
sequencing the product obtained from the preceding step to generate sequencing reads;
analysing the sequencing reads to determine the structure of the RNA molecule of the gene; and
evaluating the amount of sequencing reads to determine the expression of the gene.

16. The method of claim 1, wherein the method further comprises characterising a cell by:
determining the structure of RNA molecules in the cell.

17. The method of claim 1, wherein the method further comprises classifying cells into one or more cell populations by:
determining the structure of RNA molecules in each cell; and
classifying the cells into one or more cell populations based on similarity in the structure of their RNA molecules.

18. (canceled)

19. A kit for determining a structure of RNA molecules, the kit comprising:
a modifying agent comprising 2-methylnicotinic acid imidazolide (NAI) or derivatives thereof for modifying the RNA molecules;
a reverse transcription medium comprising manganese; and
optionally, a Moloney murine leukemia virus (MMLV) reverse transcriptase, further optionally a genetically modified MMLV reverse transcriptase.

20. The kit of claim 19, the kit further comprising:
a single medium for fragmentation of the RNA molecules and annealing of a primer to the RNA molecules for reverse transcription, wherein the single medium is substantially free of magnesium, optionally wherein the single medium comprises deoxyribonucleotide triphosphates (dNTPs).

21. The method of claim 1, wherein the method further comprises classifying cells into one or more cell populations by:
determining the structure of RNA molecules in each cell; and
classifying the cells into one or more cell populations based on similarity in the structure of their RNA molecules,
or wherein the method is a method of classifying cells into different cell types and/or different stages of development.

22. The method according to claim 1, wherein the fragmenting step is carried out in the same vessel as the reverse transcribing step and wherein the method further comprises purifying the amplicons, further optionally purifying the amplicons for at least two times.

23. The method according to claim 1, wherein fragmenting the modified RNA molecules comprises heating the modified RNA molecules, optionally wherein the modified RNA molecules is heated in the presence of deoxynucleoside triphosphate (dNTP).
